(19) United States
(12) Patent Application Publication
(10) Pub. No.: US 2011/0218585 A1
(43) Pub. Date: Sep. 8, 2011

(54) APPARATUS AND METHODS FOR BONE REPAIR

(76) Inventors: Todd A. Krinke, Buffalo, MN (US); Steve D. Kruse, St. Michael, MN (US); Kyle Taylor, Brooklyn Park, MN (US); Stefan J. Hertel, Minneapolis, MN (US); Michael P. Brenzel, St. Paul, MN (US); Paul Hindrichs, Plymouth, MN (US)

(21) Appl. No.: 13/043,330

(22) Filed: Mar. 8, 2011

Related U.S. Application Data
(60) Provisional application No. 61/311,494, filed on Mar. 8, 2010.
(60) Provisional application No. 61/378,822, filed on Aug. 31, 2010.

(51) Int. Cl. A61B 17/56

(52) U.S. Cl. 606/86 R

(57) ABSTRACT

Apparatus and methods for repairing a bone. The apparatus and methods may involve transferring a mechanical load from a first bone fragment to a second bone fragment. For example, the first bone fragment may be at the end of the bone. The second bone fragment may be in the diaphyseal region of the bone. The bone fragment at the end of the bone may be separated by a fracture from the bone fragment in the diaphyseal region of the bone. The fracture may interfere with transmission of the load from the bone fragment at the end of the bone to the bone fragment in the diaphyseal region of the bone. Transmission of the load across the fracture by the apparatus may promote healing of the fracture.

### CROSS-REFERENCE TO RELATED APPLICATIONS

This application is a nonprovisional of U.S. Provisional Applications Nos. 61/311,494, filed on Mar. 8, 2010, and 61/378,822, filed on Aug. 31, 2010, both of which are hereby incorporated by reference in their entireties.

### FIELD OF TECHNOLOGY

Aspects of the disclosure relate to providing apparatus and methods for repairing bone fractures. In particular, the disclosure relates to apparatus and methods for repairing bone fractures utilizing a device that is inserted into a bone.

### BACKGROUND

Bone fracture fixation may involve using a structure to counteract or partially counteract forces on a fractured bone or associated bone fragments. In general, fracture fixation may provide longitudinal (along the long axis of the bone), transverse (across the long axis of the bone), and rotational (about the long axis of the bone) stability. Fracture fixation may also preserve normal biologic and healing function.

Bone fracture fixation often involves addressing loading conditions, fracture patterns, alignment, compression force, and other factors, which may differ for different types of fractures. For example, midshaft fractures may have ample bone material on either side of the fracture in which anchors may be driven. End-bone fractures, especially on the articular surface may have thin cortical bone, soft cancellous bone, and relatively fewer possible anchoring locations. Typical bone fracture fixation approaches may involve one or both of: (1) a device that is within the skin (internal fixation); and (2) a device that extends out of the skin (external fixation).

Internal fixation approaches often involve a plate that is screwed to the outside of the bone.

Plates are often characterized by relatively invasive surgery, support of fractured bone segments from one side outside of bone, and screws that anchor into the plate and the bone.

Multi-segment fractures, of either the midshaft or end-bone, may require alignment and stability in a manner that generates adequate fixation in multiple directions. Implants may be used to treat midshaft fractures and end-bone fractures.

Proper location, size, shape, orientation and proximity to bone fragments and anatomical features, among other factors, may increase the therapeutic effectiveness of the implant.

It would therefore be desirable to provide apparatus and methods for repairing a bone.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows illustrative apparatus in accordance with principles of the invention along with illustrative anatomy in connection with which the invention may be practiced.

FIG. 2 shows a view, taken along lines 2-2 (shown in FIG. 1), of the apparatus and anatomy shown in FIG. 1.

FIG. 3 shows a view, taken along lines 3-3 (shown in FIG. 1), of the apparatus and anatomy shown in FIG. 1.

FIG. 4 shows a view, taken along lines 4-4 (shown in FIG. 1), of the apparatus and anatomy shown in FIG. 1.

FIG. 5 shows the anatomy shown in FIG. 1.

FIG. 6 shows a portion of the apparatus shown in FIG. 1 and the anatomy shown in FIG. 1.

FIG. 7 shows another illustrative apparatus in accordance with principles of the invention.

FIG. 8 shows a partial cross-sectional view, taken along lines 8-8 (shown in FIG. 7), of the apparatus shown in FIG. 7.

FIG. 9 shows yet another illustrative apparatus in accordance with principles of the invention.

FIG. 10 shows still other illustrative apparatus in accordance with principles of the invention along with illustrative anatomy in connection with which the invention may be practiced.

FIG. 11 shows a view, taken along lines 11-11 (shown in FIG. 10) of the apparatus and anatomy shown in FIG. 10.

FIG. 12A shows still other illustrative apparatus in accordance with principles of the invention along with illustrative anatomy in connection with which the invention may be practiced.

FIG. 12B shows still other illustrative apparatus in accordance with principles of the invention.

FIG. 13 shows a view, taken along lines 13-13 (shown in FIG. 12A), of the apparatus and anatomy shown in FIG. 12A.

FIG. 14 shows still another illustrative apparatus in accordance with principles of the invention.

FIG. 15 shows still other illustrative apparatus in accordance with principles of the invention.

FIG. 16 shows a view, taken along lines 16-16 (shown in FIG. 15), of the apparatus shown in FIG. 15.

FIG. 17 shows still another illustrative apparatus in accordance with principles of the invention along with illustrative anatomy in connection with which the invention may be practiced.

FIG. 18 shows apparatus that may be used in conjunction with apparatus in accordance with the principles of the invention along with anatomy in connection with which the invention may be practiced.

FIG. 19 shows still other illustrative apparatus in accordance with principles of the invention.

FIG. 20 shows still other illustrative apparatus in accordance with principles of the invention

FIG. 21 shows still other illustrative apparatus in accordance with principles of the invention along with illustrative anatomy in connection with which the invention may be practiced.

FIG. 22 shows still other illustrative apparatus in accordance with principles of the invention along with illustrative anatomy in connection with which the invention may be practiced.

FIG. 23 shows still other illustrative apparatus in accordance with principles of the invention.

FIG. 24 shows still other illustrative apparatus in accordance with principles of the invention.

FIG. 25 shows a view, taken along lines 25-25 (shown in FIG. 24), of the apparatus shown in FIG. 24.

FIG. 26 shows schematically an illustrative embodiment of the apparatus of FIG. 25 in a state that is different from the state shown in FIG. 25.

FIG. 27 shows other illustrative anatomy in connection with which the invention may be practiced.

FIG. 28 shows yet other illustrative anatomy in connection with which the invention may be practiced.

### DETAILED DESCRIPTION OF THE INVENTION

Apparatus and methods for repairing a bone are provided. The apparatus and methods may involve transferring a mechanical load from a first bone fragment to a second bone fragment. The first and second bone fragments may be in any regions of the bone. For example, the first bone fragment may be at the end of the bone. The second bone fragment may be in the diaphyseal region of the bone.

The bone fragment at the end of the bone may be separated by a fracture from the bone fragment in the diaphyseal region of the bone. The fracture may interfere with transmission of the load from the bone fragment at the end of the bone to the bone fragment in the diaphyseal region of the bone. The transmission of the load across the fracture may interfere with healing of the fracture. The transmission of the load across the fracture may cause damage to bone fragments adjacent the fracture. The bone fragment in the diaphyseal region of the bone may have sufficient mechanical integrity to transmit the load along to other skeletal structures.

The apparatus may be delivered to an interior region of the bone via the one or more access holes. The access hole or holes may be provided by a bone drill, a bone saw or any other suitable device, such as one or more of the devices that are shown and described in U.S. Patent Application Publication No. 2009/0182336A1, U.S. patent application Ser. No. 13/009,657, U.S. patent application Ser. No. 13/043,190, filed on Mar. 8, 2011, or U.S. Provisional Patent Application No. 61/450,112, filed on Mar. 7, 2011, all of which are hereby incorporated by reference herein in their entireties.

The interior region may be prepared by any suitable bone cavity preparation device such as one or more of the devices that are shown and described in the aforementioned patent publication and applications.

The apparatus and methods may involve the expansion of devices in the interior region of the bone. The expansion may involve any suitable expansion mechanism or technique, such as one or more of the mechanisms and techniques that are shown and described in the aforementioned patent publication and applications.

The bone may define a bisecting longitudinal plane that bisects the bone along a longitudinal axis of the bone.

The apparatus may include, and the methods may involve, a bone truss and the methods may involve a bone truss. The truss may include elongated members. Each of the elongated members may be inserted substantially fully into a bone and, then, locked to another of the elongated members. The elongated members may define a triangular region inside the bone.

The elongated members may include a subchondral member. The elongated members may include a first diagonal member. The first diagonal member may be configured to span from a first subchondral position to a second diaphyseal position. The second diaphyseal position may be diagonally across the longitudinally bisecting plane from the first subchondral member.

The elongated members may include a second diagonal member. The second diagonal member may be configured to span from a second subchondral position to a first diaphyseal position. The first diaphyseal position may be diagonally across the longitudinally bisecting plane from the second subchondral position.

The subchondral member may be tubular.

The first diagonal member may be tubular.

The elongated members may include a diaphyseal member. The diaphyseal member may be configured to span from the first diaphyseal position to the second diaphyseal position.

The subchondral member may include a subchondral tubular structure. The subchondral tubular structure may include a cell that is configured to receive a bone anchor. The cell may be one of a plurality of cells, each of which being configured to receive a bone anchor.

The cell may be an open cell. An open cell may have a diameter that is sufficient for receipt of a portion of a bone anchor. The cell may be a closed cell. A closed cell may have a diameter that is insufficient for receipt of a portion of a bone anchor. A closed cell may deform such that its diameter enlarges in response to stress from an anchor. The stress may open the closed cell so that the cell can receive the anchor.

The subchondral tubular structure may be expandable.

The first diagonal member may include a diagonal tubular structure. The diagonal tubular structure may be configured to be joined at the first subchondral position directly to the subchondral tubular structure.

The diaphyseal member may include a diaphyseal tubular structure. The diaphyseal tubular structure may include a cell that is one of a plurality of cells, each cell being configured to receive a bone anchor. The cell may be an open cell. The cell may be a closed cell.

The diaphyseal tubular structure may be expandable. The diaphyseal tubular structure may be configured to be joined at the second diaphyseal position directly to the first diagonal member.

The second diagonal member may be configured to transmit compressive force, in an outward radial direction relative to a longitudinal axis of the bone, to the first diaphyseal position. The diaphyseal member may be configured to transmit tensile force, in an inward radial direction relative to the longitudinal axis, to the first diaphyseal position.

The second diagonal member and the diaphyseal member may be configured such that the outward radial force has a magnitude that is approximately the same as a magnitude of the inward radial force.

The first diagonal member and the second diagonal member may form a node. The first diagonal member may be configured to transmit compressive force from the first subchondral position to the node. The node may be configured to transmit a first portion of the compressive force along the first diagonal member to the second diaphyseal position. The node may be configured to transmit a second portion of the compressive force along the second diagonal member to the first diaphyseal position.

The first diagonal member and the second diagonal member may be configured to form a node. The second diagonal member may be configured to transmit compressive force from the first subchondral position to the node. The node may be configured to transmit a first portion of the compressive force along the first diagonal member to the second diaphyseal position. The node may be configured to transmit a second portion of the compressive force along the second diagonal member to the first diaphyseal position.

The apparatus may include, and the methods may involve, a tubular implant for the bone.

The tubular implant may include a first end that is configured to couple subchondrally to the bone at a loading position; and a second end that is configured to couple to the bone at a diaphyseal position. The diaphyseal position may be across the longitudinally bisecting plane of the bone from the loading position.

The second end may terminate at a surface that is oblique to a length of the implant. The surface may be substantially parallel to a diaphyseal surface of the bone. The diaphyseal surface may be an outer cortical surface of the bone. The diaphyseal surface may border an access hole in the cortical bone.

The tubular implant may include an inner tubular surface. The second end may include, in the inner tubular surface, an anchor receiving feature. The anchor receiving feature may be configured to receive an anchor. The anchor may be configured to penetrate cortical bone adjacent the anchor receiving feature and cortical bone that is across the longitudinally bisecting plane of the bone from the anchor receiving feature.

The inner tubular surface may define, at the second end, a pocket that accommodates, between an inner wall of the cortical bone and an outer wall of the cortical bone, a portion of a head of the anchor.

The tubular implant may include a tubular wall. The tubular wall may define a first elongated window and a second elongated window. The second elongated window may be opposite the first elongated window. Each of the first and second elongated windows may be configured to receive a body of an anchor and engage an engagement feature of the anchor.

The first and second elongated windows are configured to cooperatively brace the anchor at an angle relative to the tubular implant, the angle being determined by an angle at which the anchor enters the first elongated window.

The tubular implant may be expandable. The tubular implant may include a web of anchor receiving cells.

The apparatus may include, and the methods may involve, apparatus for treating an end of a bone.

Some of the methods may include preparing an elongated subchondral cavity that is transverse to a longitudinal axis of the bone; expanding a web of anchor receiving cells in the subchondral cavity; and engaging the web with an anchor that is anchored to a portion of the bone.

The expanding may include expanding a web that has a central axis and a diameter that varies along the central axis.

The apparatus may include, and the methods may involve, an anchor-receiving bone support. The bone support may include a tube wall. The tube wall may define a first elongated window. The tube wall may define a second elongated window. The second elongated window may be opposite the first elongated window. Each of the first and second elongated windows may be configured to be traversed by a body of an anchor. Each of the first and second elongated windows may be configured to be engaged by an engagement feature of the anchor.

The anchor may be a screw. The body may be a screw root. The engagement feature may be a screw thread.

The support the first and second elongated windows may be configured to cooperatively brace the anchor at an angle relative to the tubular implant. The angle may be an angle that is in a range from (a) perpendicular to the implant to (b) an angle that is defined by an outer diameter of the tubular implant, a radius of the anchor and a longitudinal displacement between an end of the first elongated window and an end of the second elongated window.

The tube wall may be a first tube wall. The support may include a second tube wall. The second tube wall may include a transverse slot. The transverse slot may be configured to be moved to different positions along the first and second elongated windows. The transverse slot may be configured to be traversed by a body of the anchor and engaged by an engagement feature of the anchor.

The first tube wall may be nested inside the second tube wall. The second tube wall may be nested inside the first tube wall.

The first elongated window, the second elongated window and the transverse slot may be configured to cooperatively brace the anchor against rotation relative to a longitudinal axis of the first tube wall. The first elongated window, the second elongated window and the transverse slot may be configured to cooperatively brace the anchor against rotation relative to a longitudinal axis of the second tube wall.

The apparatus may include, and the methods may involve, a cutting tubular bone support. The cutting tubular bone support may include a tubular web of anchor receiving cells; and a ring of saw teeth. The ring of saw teeth may be configured to saw an access hole. The access hole may be used for delivery of the bone support to the bone interior region.

The cutting tubular bone support may be configured to be locked into a bone support truss after being delivered to the intramedullary space.

The cutting tubular support may include solid tube that is longitudinally contiguous with the tubular web.

The apparatus may include, and the methods may involve, a bone anchor substrate. The bone anchor substrate may include a first elongated member comprising first anchor receiving features; a second elongated member comprising second anchor receiving features; and a coupling that is configured to resist distancing of the second elongated member from the first elongated member in response to a transverse force.

The bone anchor substrate may include a first elongated member including a first web of anchor receiving features; and a second elongated member including a second web of anchor receiving features. The second elongated member may be configured to be deployed alongside the first elongated member in an interior region of a bone.

If an elongated member is expandable, a delivery state diameter may be a collapsed diameter. If an elongated member is not expandable, the delivery state diameter may be a static diameter.

The first elongated member may have a first delivery state diameter. The first elongated member may be configured to be delivered to the interior region through a guide tube that has an inner diameter. The second elongated member may have a second delivery state diameter. The second elongated member may be configured to be delivered to the interior region through the guide tube. A sum of the first and second delivery state diameters may be greater than the inner diameter. The first and second elongated members may be sequentially deployed in the interior region. The sum of the first and second delivery state diameters may be less than the inner diameter. The first and second elongated members may be concurrently deployed in the interior region.

The first elongated member may have a first longitudinal axis. The second elongated member may have a second longitudinal axis. The first and second elongated members may be deployed in the interior region such that the first and second longitudinal axes are substantially parallel.

The first and second elongated members may be members of a group of elongated members. The bone anchor substrate may have a central axis. The central axis may be central to the group of elongated members.

The first elongated member may have a first longitudinal axis. The second elongated member may have a second longitudinal axis. If the first and second elongated members are expandable, when the first and second elongated members are expanded in the interior region, the first and second longitudinal axes may be substantially conically arranged about the central axis.

The first web may include a first anchor receiving feature. The second web may include a second anchor receiving feature. The first and second anchor receiving features may be sufficiently aligned with each other to engage a bone anchor that penetrates a fragment of the bone.

Each member of the group may be configured to be deployed alongside another member of the group in the interior region of the bone.

A first member of the group may be configured to transmit load from a first bone fragment to a second bone fragment via a second member of the group. The first and second members of the group may communicate load via surface contact between the first and second members. The first and second members of the group may communicate load via a coupling. The first and second members of the group may communicate load via an anchor.

The coupling may be configured to resist the distancing during traversal of the first elongated member and the second elongated member by a bone anchor.

The coupling may be configured to resist the distancing during loading of the first elongated member and the second elongated member by a bone anchor.

One or both of the first elongated member and the second elongated member may be expandable.

One or both of the first elongated member and the second elongated member may have a radius that varies along the length of the elongated member.

The first anchor receiving features may include an open cell in a web of open cells.

The first anchor receiving features may include a closed cell in a web of closed cells.

The first anchor receiving features may include a tubular portion. The tubular portion may define an anchor receiving slot. The tubular portion may define an anchor receiving hole.

The bone anchor substrate may include, in addition to the first elongated member and the second elongated member, a plurality of elongated members. The coupling may be configured to resist distancing of each of the plurality of elongated members, the first elongated member and the second elongated member from another of the plurality of elongated members, the first elongated member and the second elongated member.

One or more surfaces of the apparatus may be coated with agents that promote bone ingrowth. The agents may include calcium phosphate, heat treated hydroxylapatite, Basic fibroblast growth factor (bFGF)-coated hydroxyapatite, hydroxyapatite/tricalcium phosphate (HA/TCP), and other suitable agents, including one or more of those listed in Table 1.

One or more surfaces of the apparatus may be coated with agents that inhibit or prohibit bone ingrowth. Such surfaces may include impermeable and other materials such as one or more of those listed in Table 1.

One or more surfaces of the apparatus may be coated with agents that may elute therapeutic substances such as drugs.

The apparatus and portions thereof may include any suitable materials. Table 1 lists illustrative materials that may be included in the apparatus and portions thereof.

TABLE 1
Materials

| Category | Type | Material |
|---|---|---|
| Metals | Nickel titanium alloys | Nitinol |
| | Stainless steel alloys | 304 |
| | | 316L |
| | | BioDur ® 108 Alloy |
| | | Pyromet Alloy ® CTX-909 |
| | | Pyromet ® Alloy CTX-3 |
| | | Pyromet ® Alloy 31 |
| | | Pyromet ® Alloy CTX-1 |
| | | 21Cr—6Ni—9Mn Stainless |
| | | 21Cr—6Ni—9Mn Stainless |
| | | Pyromet Alloy 350 |
| | | 18Cr—2Ni—12Mn Stainless |
| | | Custom 630 (17Cr—4Ni) |
| | | Stainless |
| | | Custom 465 ® Stainless |
| | | Custom 455 ® Stainless Custom |
| | | 450 ® Stainless |
| | | Carpenter 13-8 Stainless |
| | | Type 440C Stainless |
| | Cobalt chromium alloys | MP35N |
| | | Elgiloy |
| | | L605 |
| | | Biodur ® Carpenter CCM alloy |
| | Titanium and titanium | Ti—6Al—4V/ELI |
| | alloys | Ti—6Al—7Nb |
| | | Ti—15Mo |
| | Tantalum | |
| | Tungsten and tungsten | |
| | alloys | |
| | Pure Platinum | |
| | Platinum- Iridium | |
| | alloys | |
| | Platinum -Nickel | |
| | alloys | |
| | Niobium | |
| | Iridium | |
| | Conichrome | |
| | Gold and Gold alloys | |
| Absorbable | | Pure Iron |
| metals | | magnesium alloys |
| Polymers | | Polyetheretherketone (PEEK) |
| | | polycarbonate |
| | | polyolefin's |
| | | polyethylene's |
| | | polyether block amides |
| | | (PEBAX) |
| | | nylon 6 |
| | | 6-6 |
| | | 12 |
| | | Polypropylene |
| | | polyesters |
| | | polyurethanes |
| | | polytetrafluoroethylene (PTFE) |
| | | Poly(phenylene sulfide) (PPS) |
| | | poly(butylene terephthalate) |
| | | PBT |
| | | polysulfone |
| | | polyamide |
| | | polyimide |
| | | poly(p-phenylene oxide) PPO |
| | | acrylonitrile butadiene |
| | | styrene (ABS) |
| | | Polystyrene |
| | | Poly(methyl methacrylate) |
| | | (PMMA) |
| | | Polyoxymethylene (POM) |
| | | Ethylene vinyl acetate |
| | | Styrene acrylonitrile resin |
| | | Polybutylene |
| Membrane | | Silicone |
| materials | | Polyether block amides |
| | | (PEBAX) |
| | | Polyurethanes |
| | | Silicone polyurethane |
| | | copolymers |
| | | Nylon |
| | | Polyethylene terephthalate |
| | | (PET) |
| | | Goretex ePTFE |
| | | Kevlar |
| | | Spectra |
| | | Dyneena |
| | | Polyvinyl chrloride (PVC) |
| Absorbable | | Poly(glycolic acid) (PGA) |
| polymers | | Polylactide (PLA), |
| | | Poly(ε-caprolactone), |
| | | Poly(dioxanone) |
| | | Poly(lactide-co-glycolide) |
| Radiopaque | | Barium sulfate |
| materials | | Bismuth subcarbonate |
| Biomaterials | Collagen | Bovine, porcine, ovine, amnion |
| | | membrane |
| Bone growth | | Demineralized bone matrix |
| factors | | Bone morphogenic proteins |
| | | (BMP) |
| | | Calcium phosphate |
| | | Heat-treated |
| | | hydroxylapapatite |
| | | Basic fibroblast growth factor |
| | | (bFGF) -coated hydroxyapaptite |
| | | Hydroxyapaptite/tricalcium |
| | | phosphate (HA/TCP |
| Anti- | | |
| microbial | | |
| Coatings | | |

The apparatus may be provided as a kit that may include one or more of a structural support, an anchoring substrate, a central axis member, an anchor, a delivery instrument and associated items.

Apparatus and methods in accordance with the invention will be described in connection with the FIGS.

The FIGS. show illustrative features of apparatus and methods in accordance with the principles of the invention. Apparatus and methods of the invention may involve some or all of the illustrative features. The features are illustrated in the context of selected embodiments. It is to be understood that other embodiments may be utilized and structural, functional and procedural modifications may be made without departing from the scope and spirit of the present invention. The steps of illustrative methods may be performed in an order other than the order shown or described herein. Some embodiments may omit steps shown or described in connection with the illustrative methods. Some embodiments may include steps that are not shown or described in connection with the illustrative methods. It will be understood that features shown in connection with one of the embodiments may be practiced in accordance with the principles of the invention along with features shown in connection with one or more other embodiments.

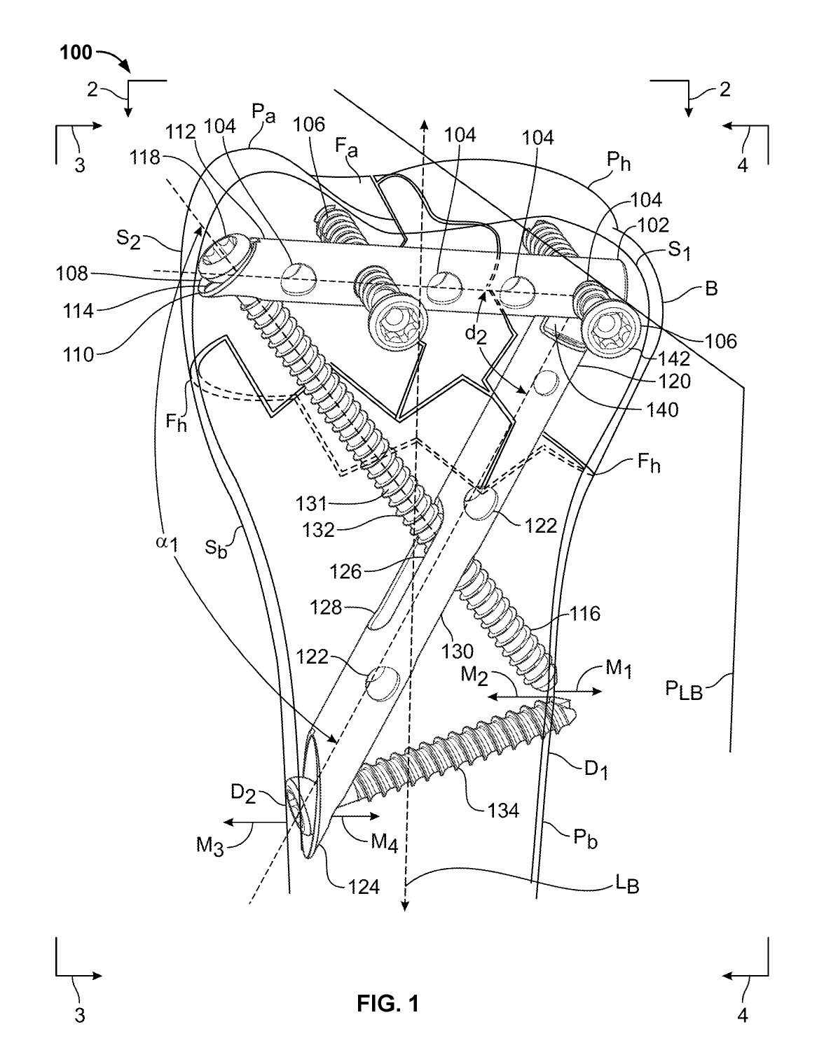

FIG. 1 shows illustrative truss 100 in bone B. Bone truss 100 may be used to fragments of a broken bone relative to each other. In FIG. 1, bone B is illustrated as including three fragments: P_{b}, Pₕ and Pₐ, which are separated by fractures Fₕ and Fₐ. Truss 100 may be used in connection with two-part fractures, three-part fractures or fracture having more than three parts.

Truss 100 may include subchondral member 102. Subchondral member 102 may be used to support one or more bone fragments such as Ph and Pₐ. Subchondral member 102 may include one or more anchor receiving features such as anchor receiving features 104. Anchors such as anchors 106 may secure fragments Pₕ and Pₐ to subchondral member 102.

Subchondral member 102 may include mitered surface 108. Mitered surface 108 may be angled to conform to surface S_{b} of bone B. Mitered surface 108 may define “scoop” 110 at 112 of subchondral member 102. Scoop 110 may conform to an access hole (not shown) in bone B. The access hole may be angled relative to surface S_{b}. Scoop 110 may include anchor receiving feature 114.

Anchor receiving feature 114 may face an inner wall (not shown) of the access hole such that diagonal anchor 116 may be driven through anchor receiving feature 114 into cortical bone that surrounds the access hole. Scoop 110 may define in the cortical bone a pocket for receiving part or all of anchor head 118 of diagonal anchor 116.

Subchondral member 102 may span across longitudinal bisecting plane Plb from subchondral position S1 to subchondral position S₂.

Truss 100 may include diagonal member 120. Diagonal member 120 may span across longitudinal bisecting plane P_{lb} from subchondral position S₂ to diaphyseal position D₂.

Diagonal member 120 may be used to transmit load from an end bone fragment such as Pₕ to a long bone fragment such as P_{b}.

Diagonal member 120 may include one or more anchor receiving features such as anchor receiving features 122. Diagonal member 120 may be fixed to subchondral member 102 at subchondral position S₂ by any suitable technique. For example, diagonal member 120 may be pinned to subchondral member 102 by anchor 106. Angle α₂ may be selected for proper positioning of diagonal member 120 at diaphyseal position D₂.

Diagonal member 120 may include scoop 124. Scoop 124 may have one or more features in common with scoop 110.

Diagonal anchor 116 may be a diagonal member of truss 100. Diagonal anchor 116 may span across longitudinal bisecting plane P_{lb} from subchondral position S₂ to diaphyseal position D.

Diagonal anchor 116 may be used to transmit load from an end bone fragment such as Pₐ to a long bone fragment such as P_{b}.

Diagonal anchor 116 may intersect with diagonal member 120 to form node 126. Node 126 may distribute load from subchondral member 102 to both diaphyseal position D₁ (along diagonal anchor 116) and diaphyseal position D₂ (along diagonal member 120).

Diagonal member 120 may include slot 128 and slot 130 (not shown) opposite slot 128. Slot 128 may have a width that is large enough to pass root 131 of diagonal anchor 116, but small enough to engage thread 132 of diagonal anchor 116. Slot 130 may have a width that is large enough to pass root 131 of diagonal anchor 116, but small enough to engage thread 132 of diagonal anchor 116. Diagonal anchor 116 may thus be retained by both slots 128 and 130. Slot 130 may have a width that is large enough to pass both root 131 of diagonal anchor 116 and thread 132 of diagonal anchor 116. When diagonal anchor 116 is retained by both slots 128 and 130, diagonal member 116 may resist rotation in directions α₁ and −α₁ to a greater extent than when diagonal anchor 116 is retained by only one of slots 128 and 130.

Diaphyseal anchor 134 may span across longitudinal bisecting plane P_{lb} from diaphyseal position D₂ to diaphyseal position D.

When truss 100 is loaded at one or more bone fragments such as fragment Ph and fragment Pₐ, diagonal anchor 116 may exert radially outward force M₁ at diaphyseal position D₁. Diagonal member 120 may exert radially outward force M₃ at diaphyseal position D₂. Diaphyseal anchor 134 may partially or wholly balance radially outward forces M₁ and M₃ by exerting radially inward forces M₂ and M₄ at diaphyseal positions D₁ and D₂, respectively.

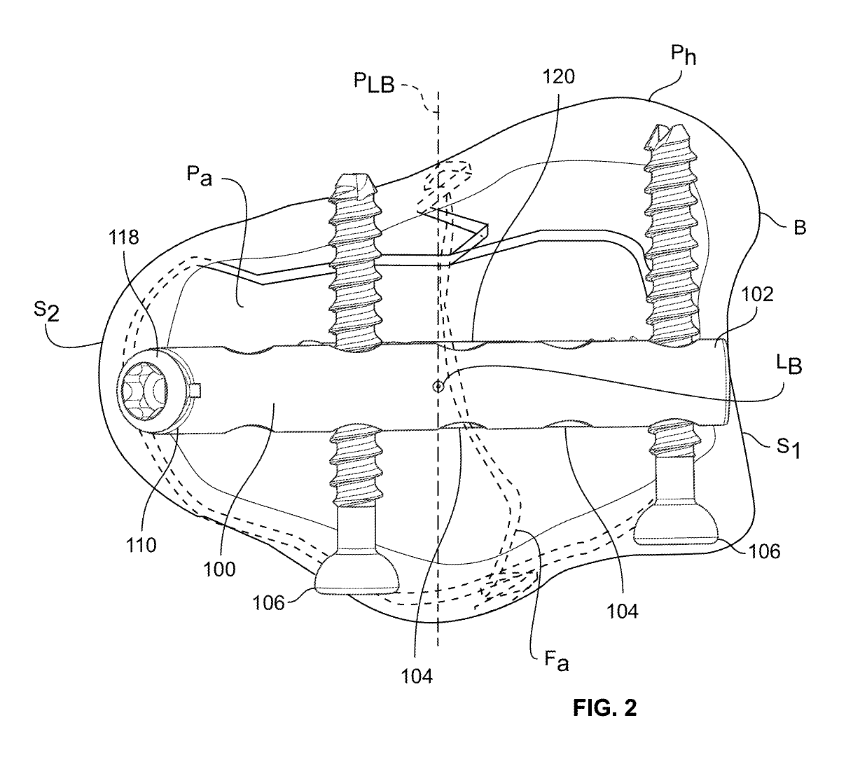

FIG. 2 shows a view taken along lines 2-2 (shown in FIG. 1) of truss 100 in bone B.

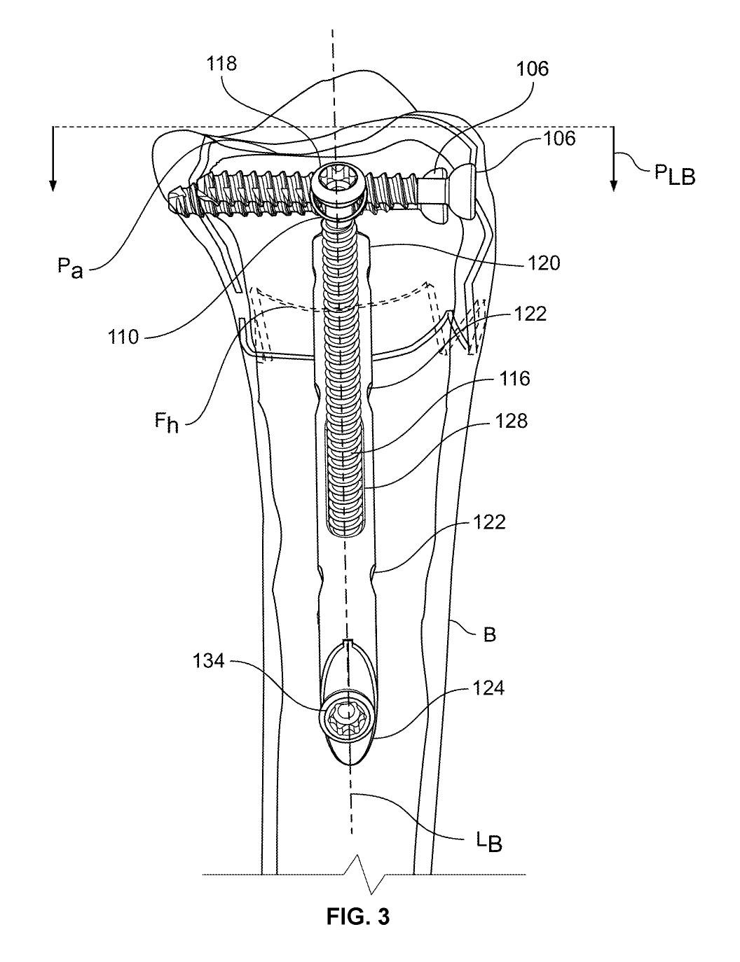

FIG. 3 shows a view taken along lines 3-3 (shown in FIG. 1) of truss 100 in bone B.

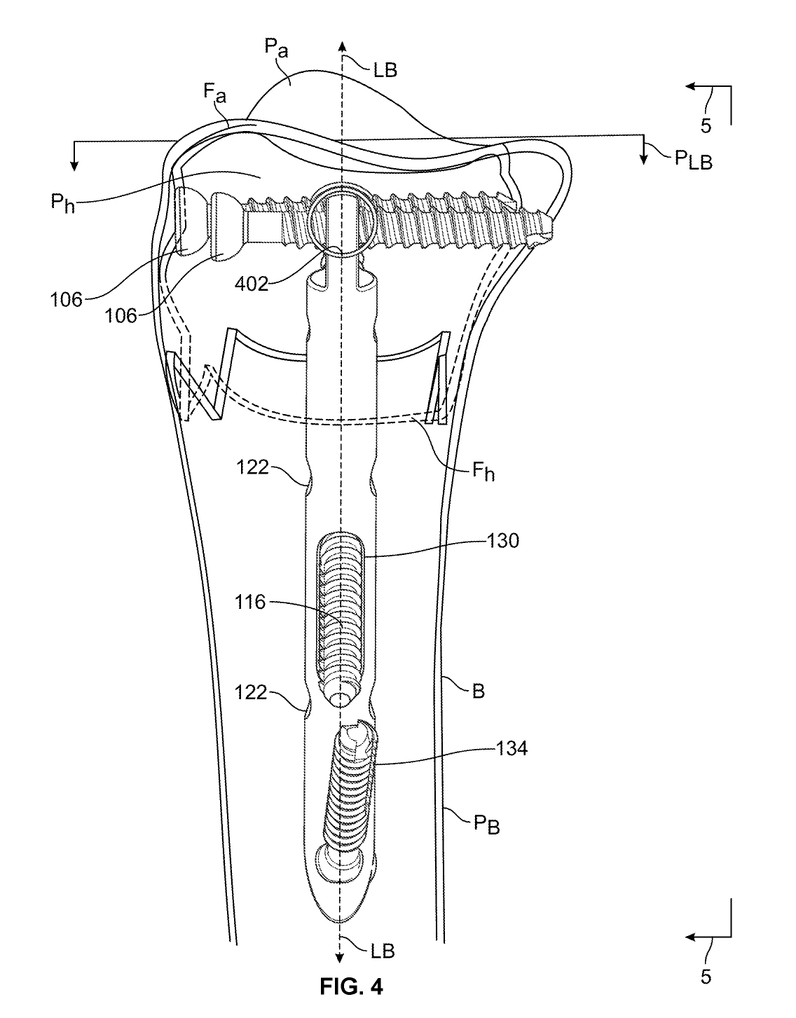

FIG. 4 shows a view taken along lines 4-4 (shown in FIG. 1) of truss 100 in bone B.

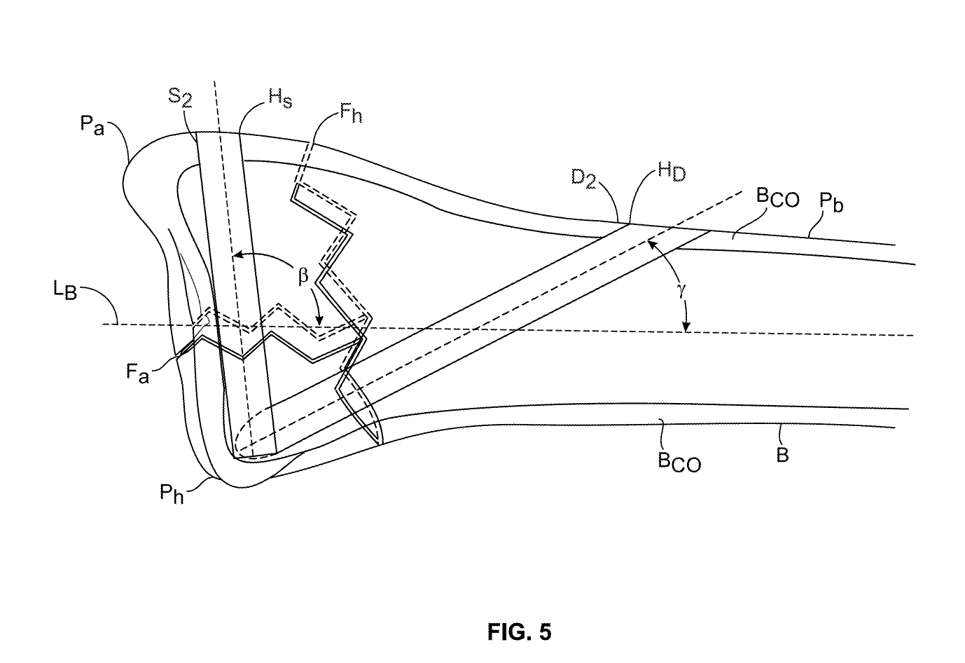

FIG. 5 shows a view taken along lines 5-5 (shown in FIG. 4) of illustrative subchondral access hole HS and diagonal access hole HD in bone B. Access hole HS may be drilled at angle β to bone axis LB. Access hole HD may be drilled at angle γ to bone axis L. Any suitable methods for drilling or sawing the holes may be used, including as such methods that are shown and described in U.S. Patent Application Publication No. 2009/0182336A1 or U.S. patent application Ser. No. 13/009,657.

Cortical bone BCO at diaphyseal position D2 may provide a foundation for scoop 124 (shown in FIG. 1). Cortical bone BCO at subchondral position S2 may provide a foundation for scoop 110 (shown in FIG. 1).

Subchondral member 102 may be inserted in hole H. Diagonal member may be inserted in hole H_{D}. Tang 140 (shown in FIG. 1), which may include an anchor pass-through, may be inserted into slot 402 (shown in FIG. 4) of subchondral member 102. Anchor 142 (shown in FIG. 1) may be inserted to pin diagonal member 120 to subchondral member 102 at subchondral position S.

A practitioner may elect to treatment certain fractures using subchondral member 102, diagonal member 120, diagonal anchor 116, and not diaphyseal anchor 134.

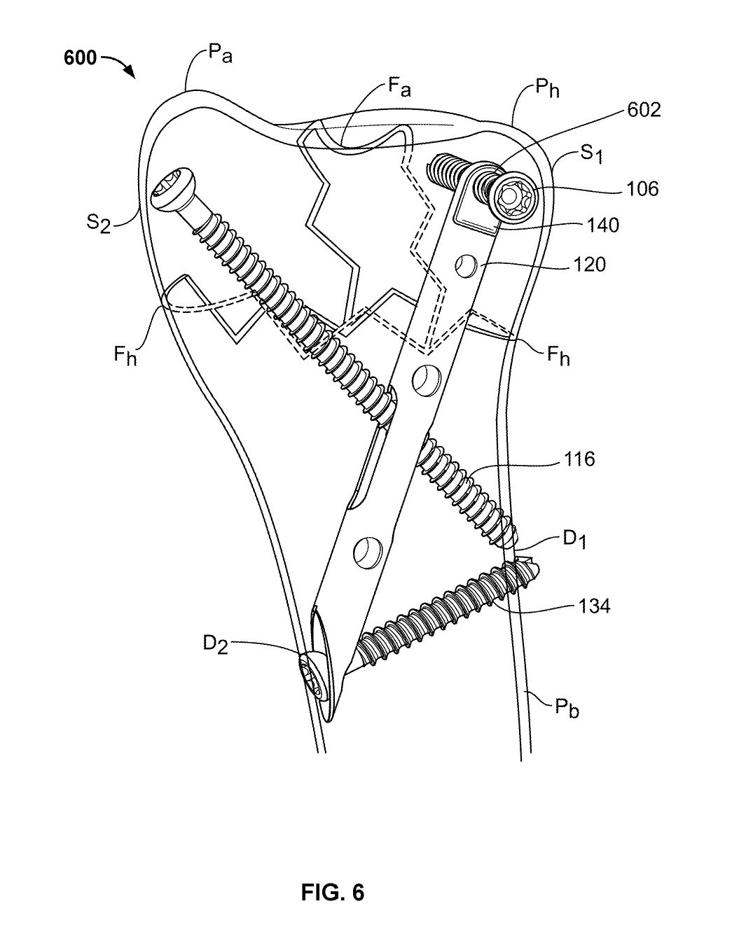

FIG. 6 shows illustrative arrangement 600 of components of truss 100. A practitioner may elect to use arrangement 600 to treat certain fractures. Arrangement 600 may include diagonal member 120, diagonal anchor 116 and diaphyseal anchor 134. Anchor 106 may be received by hole 602 in tang 140.

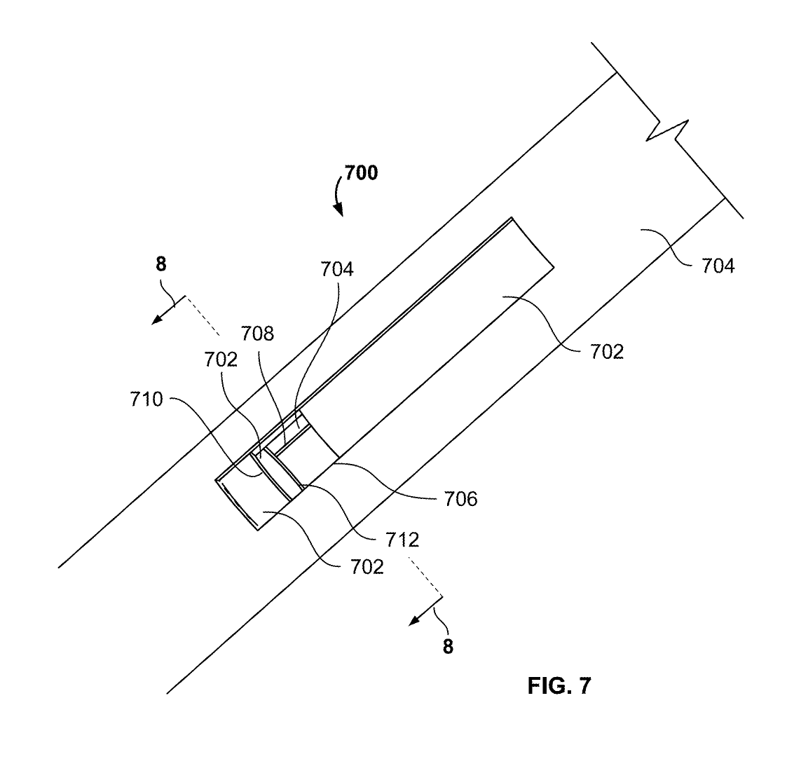

FIG. 7 shows illustrative translating anchor receiving feature 700 that may be used in conjunction with a truss element such as diagonal member 120 (shown in FIG. 1) or any other tubular truss element, such as a tubular truss element that may correspond to any of the truss elements shown in FIG. 1.

Anchor receiving feature 700 may include inner tube 702. Anchor receiving feature 700 may include outer tube 704. Outer tube 704 may include elongated window 706 and elongated window 708. Elongated window 708 may be opposite elongated window 706. Inner tube 702 may include transverse slot 710 and transverse slot 712. Transverse slot 712 may be opposite transverse slot 710.

The intersections of (a) elongated window 706 and transverse slot 710; and (b) elongated window 708 and transverse slot 712 may define two corresponding anchor vias that may be large enough to allow an anchor root such as 131 (shown in FIG. 1) to pass through and small enough to engage an anchor thread such as 132 (shown in FIG. 1).

Inner tube 702 may be slidable within outer tube 704 so that the transverse slots can be positioned at different positions relative to elongated windows 706 and 708 to accommodate anchors at the different positions. Two-tube construction may provide additional strength to a truss element.

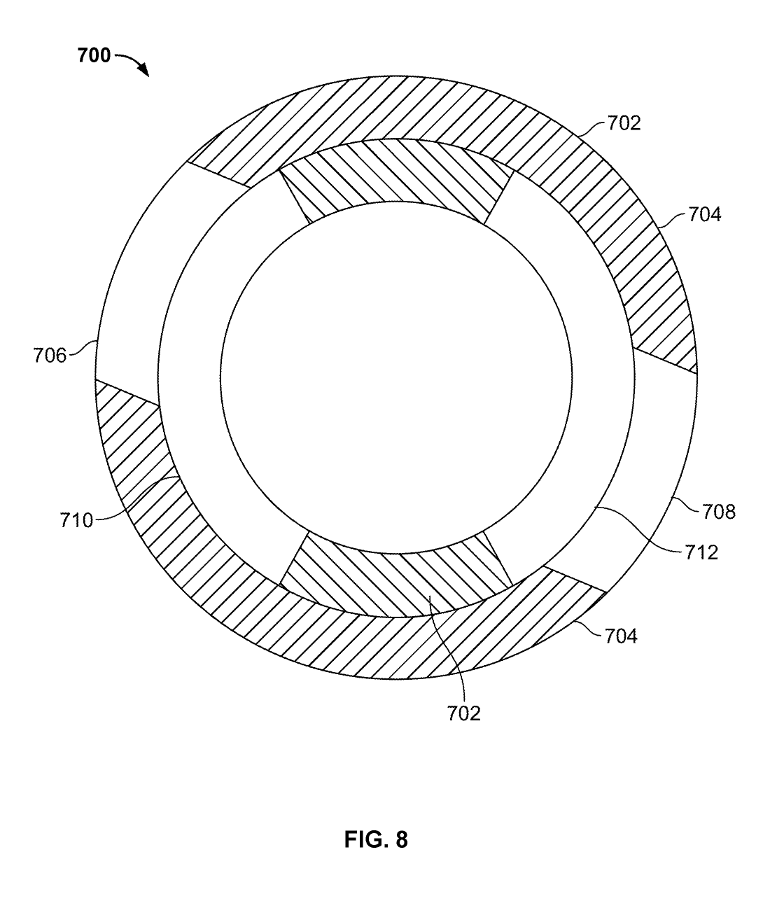

FIG. 8 shows a cross-sectional view of translating anchor receiving feature 700 taken along lines 8-8 (shown in FIG. 7).

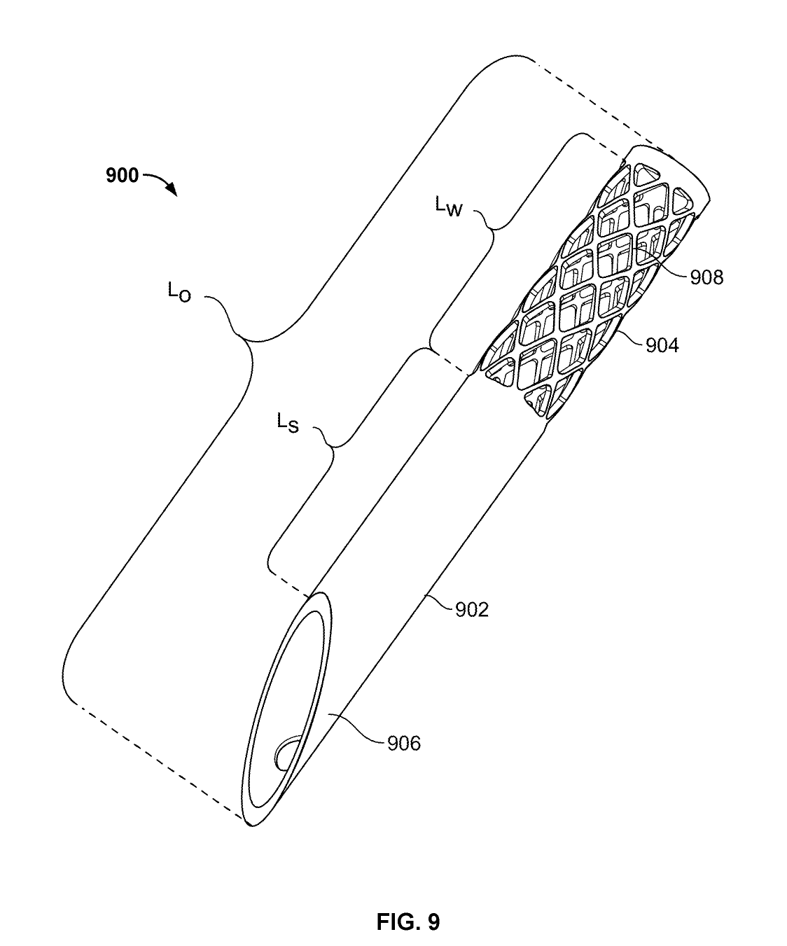

FIG. 9 shows illustrative bone support 900. Bone support 900 may be used in conjunction with one or more of the elements of truss 100 (shown in FIG. 1). Bone support 900 may be used in an orientation in bone B that corresponds to one of the orientations of the elements of truss 100.

Bone support 900 may include solid tubular portion 902. Bone support 900 may include webbed portion 904. Bone support 900 may include scoop 906. Scoop 906 may have one or more features in common with scoop 110 (shown in FIG. 1).

Bone support 900 may have overall length Lₒ. Solid tubular portion 902 may have length Lₛ. Webbed portion 904 may have length L_{w}. Lengths Ls and L_{w} may have any suitable magnitude relative to length Lₒ. Solid tubular portion 902 and webbed portion 904 may each occupy any suitable position along length Lₒ. Solid tubular portion 902 and webbed portion 904 may be present in any suitable order relative to each other.

Bone support 900 may include more than one solid tubular portion such as solid tubular portion 902. Bone support 900 may include more than one webbed portion such as webbed portion 904.

Webbed portion 904 may include cells such as cell 908. Cell 908 may receive a bone anchor such as anchor 106 (shown in FIG. 1).

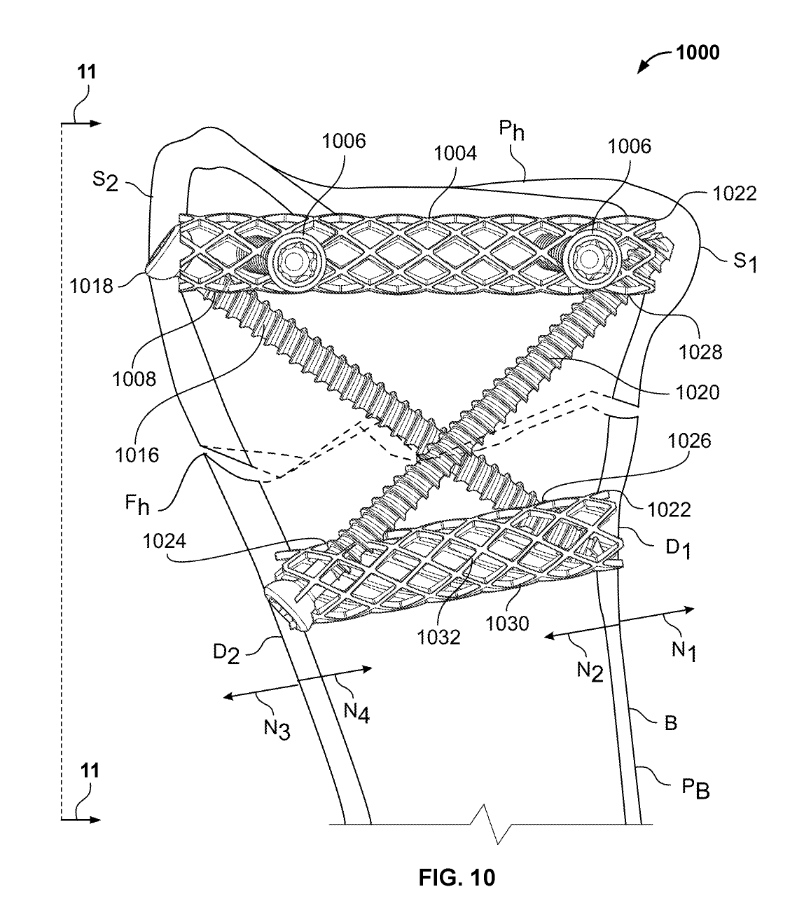

FIG. 10 shows illustrative implant 1000 in bone B. In FIG. 10, bone B is illustrated as including two fragments: P_{b} and Pₕ, which are separated by fracture Fₕ. Implant 1000 or portions thereof may be used in connection with two-part fractures, three-part fractures or fracture having more than three parts.

Implant 1000 may include subchondral member 1002. Subchondral member 1002 may be used to support one or more bone fragments such as Pₕ. Subchondral member 1002 may include web 1004. Web 1004 may include one or more anchor receiving features. Anchors such as anchors 1006 may secure fragment Ph to subchondral member 1002.

Anchor receiving feature 1008 may face an inner wall (not shown) of an access hole for subchondral member 1002 such that diagonal anchor 1016 may be driven through anchor receiving feature 1008 into cortical bone that surrounds the access hole. Subchondral member 1002 may define in the cortical bone a pocket for receiving part or all of anchor head 1018 of diagonal anchor 1016.

Subchondral member 1002 may span across longitudinal bisecting plane P_{b} (shown in FIG. 1) from subchondral position S₁ to subchondral position S₂.

Implant 1000 may include diagonal anchor 1020. Diagonal anchor 1020 may engage diaphyseal member 1022 at diaphyseal position D₂. Diagonal anchor 1020 may engage subchondral member 1002 at subchondral position S. Diagonal anchor 1020 may span across longitudinal bisecting plane P_{lb} from diaphyseal position D₂ to subchondral position S.

Diagonal anchor 1020 may engage cortical bone at diaphyseal position D₂ in a manner that is similar to that in which diagonal anchor 1016 engages cortical bone at subchondral position S₂.

Diagonal anchor 1020 may be used to transmit load from an end bone fragment such as Pₕ to a long bone fragment such as P_{b}.

Diagonal anchor 1016 may span across longitudinal bisecting plane P_{lb} from subchondral position S₂ to diaphyseal position D. Diagonal member 1016 may engage diaphyseal member 1022 at diaphyseal position D.

Diagonal anchor 116 may be used to transmit load from an end bone fragment such as Pₕ to a long bone fragment such as P_{b}.

Diagonal anchor 116 may be skewed with respect to diagonal anchor 1020.

Diaphyseal anchor 1022 may span across longitudinal bisecting plane P_{lb} from diaphyseal position D₂ to diaphyseal position D. Diaphyseal member 1022 may include web 1030. Web 1030 may include one or more anchor receiving features such as 1032.

Anchor receiving cells such as 1008, 1024, 1026 and 1028 may form joints with the diagonal anchors. The cells may be large enough to pass the roots of the anchors and small enough to be engaged by threads of the anchors. The may act like pinned joints in that the anchors may transmit moment to the subchondral and diaphyseal members ineffectively or not at all. Moment may be transferred more effectively by configuring the anchors to penetrate additional cells in the subchondral or diaphyseal members, such as cells positioned on a different aspect (e.g., spaced apart along a diameter or chord) of the respective subchondral or diaphyseal members.

When implant 1000 is loaded at one or more bone fragments such as fragment Pₕ, diagonal anchor 1016 may exert radially outward force N₁ at diaphyseal position D. Diagonal member 1020 may exert radially outward force N₃ at diaphyseal position D₂. Diaphyseal member 1022 may partially or wholly balance radially outward forces N₁ and N₃ by exerting radially inward forces N₂ and N₄ at diaphyseal positions D₁ and D₂, respectively.

One or both of subchondral member 1002 and diaphyseal member 1022 may be expandable.

One or both of subchondral member 1002 and diaphyseal member 1002 may be delivered to the interior of bone B in a manner that is analogous to the delivery of subchondral member 102 and diaphyseal member 134 (shown in FIG. 1).

A practitioner may elect to treatment certain fractures using subchondral member 1002, diagonal anchor 1020, diagonal anchor 1016, and not diaphyseal member 1022.

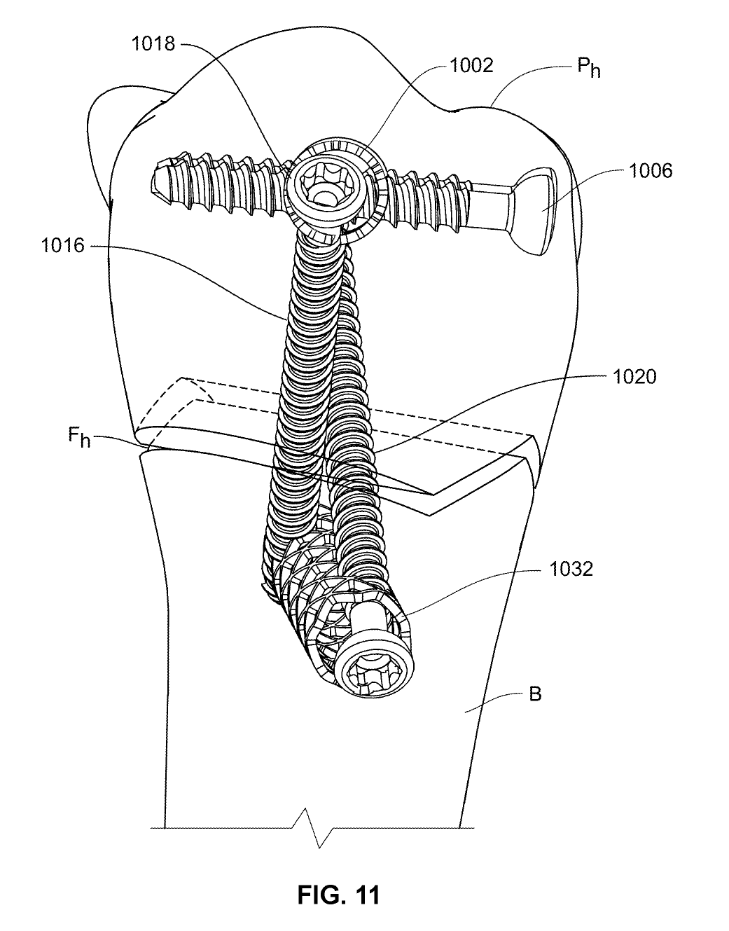

FIG. 11 shows a view taken along lines 11-11 (shown in FIG. 10) of implant 1000 in bone B.

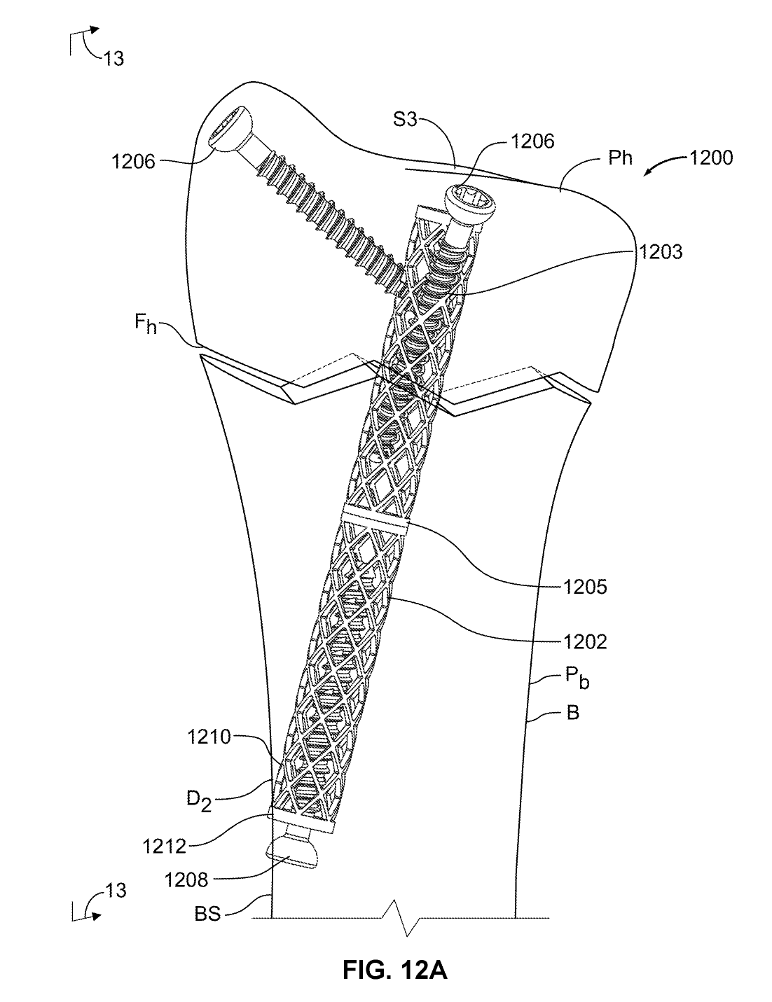

FIG. 12A shows illustrative implant 1200 in bone B. In FIG. 12, bone B is illustrated as including two fragments: Pb and Pₕ, which are separated by fracture Fₕ. Implant 1000 or portions thereof may be used in connection with two-part fractures, three-part fractures or fracture having more than three parts.

Implant 1200 may be used to support one or more bone fragments such as Pₕ. Implant 1200 include web 1202. Web 1202 may include one or more anchor receiving features such as cell 1203. Implant 1200 may include structural ring 1205. Web 1202 may be expandable distal or proximal of structural ring 1205. Web 1202 may be expandable both distal and proximal of structural ring 1205. Structural ring 1205 may not be included in implant 1202. In such embodiments, web 1202 may be expandable along the length of implant 1200.

An additional tubular web (not shown) may be provided substantially coaxially within web 1202 to provide additional anchoring strength. An additional tubular web (not shown) may be provided substantially coaxially about web 1202 to provide additional anchoring strength. Additional tubular webs (not shown) may be provided substantially coaxially about and within web 1202 to provide additional anchoring strength.

Anchors such as anchors 1206 may secure fragment Ph to implant 1202 at one or more of the cells.

Implant 1200 may span across longitudinal bisecting plane P_{lb} (shown in FIG. 1) from subchondral position S3 to diaphyseal position D₂. Implant 1200 may span from a subchondral position substantially in longitudinal bisecting plane P_{lb} (shown in FIG. 1) to diaphyseal position D₂. Implant 1200 may span from a subchondral position to a diaphyseal position without traversing plane P_{b}.

Implant 1200 may include anchor 1208. Anchor 1208 may anchor implant 1200 to cortical bone at diaphyseal position D₂. Although anchor 1208 is shown as being axially aligned with web 1202, anchor 1208 may anchor implant 1200 by penetrating cortical bone transversely to bone B at diaphyseal position D₂ and then engaging a cell at diaphyseal end 1210 of web 1202.

Implant 1200 may include a cortical bone bracket (not shown) for anchoring to cortical bone at diaphyseal position D₂. Any suitable bracket may be used. For example, the bracket may have one or more features in common with scoop 110 (shown in FIG. 1). The bracket may have an anchor receiving member that faces an inner wall (not shown) of an access hole for implant 1200 such that an anchor (not shown) be driven through the anchor receiving feature into the cortical bone that surrounds the access hole.

Anchor 1208 may be oriented axially relative to implant 1200. Anchor 1208 may engage the bracket (not shown) at diaphyseal position D2, which may be fixed to the cortical bone, and diaphyseal end 1210 of implant 1200 to secure implant 1200 to cortical bone at diaphyseal position D2. Diaphyseal end 1210 may include a tapped bushing (not shown) for engaging anchor 1208. Anchor 1208 may have appropriate threads for engaging the tapped bushing.

Implant 1200 may be used to transmit load from an end bone fragment such as Ph to a long bone fragment such as P_{b}.

Implant 1200 may be used to compress bone fragment Ph to bone fragment P_{b} at fracture F by tensioning web 1202 between anchors 1206 and 1208.

Anchor receiving cells such as 1203 may have one or more features in common with a cell such as 1008 (shown in FIG. 10).

Implant 1200 may be delivered to the interior of bone B in a manner that is analogous to the delivery of subchondral member 102 and diaphyseal member 134 (shown in FIG. 1).

FIG. 12B shows illustrative stabilizer 1220. Stabilizer 1220 may secure proximal end 1212 of implant 1200 to bone B at diaphyseal position D₂ (or at any other suitable position on bone B). Stabilizer 1220 may include elongated member 1232. Elongated member 1232 may extend from proximal end of implant 1200 (not shown) to buttress collar 1222. Elongated member 1232 may extend along the wall of the access hole through which implant 1200 is deployed. Elongated member 1232 may include longitudinal axis LEM. Longitudinal axis L_{EM} may be substantially parallel to central axis C_{H} of the hole and/or a longitudinal axis of an implant). Buttress collar 1222 may be supported at an opening of the hole. Buttress collar 1222 may include a longitudinal axis L_{BC} substantially parallel to bone surface B_{S} (shown in FIG. 12A).

Stabilizer 1220 may include an anchor receiving feature (not shown) configured to receive an anchor, such as anchor 1224, which is driven into bone surface B.

Proximal end 1212 of implant 1200 may be secured to bone B using any other suitable approach.

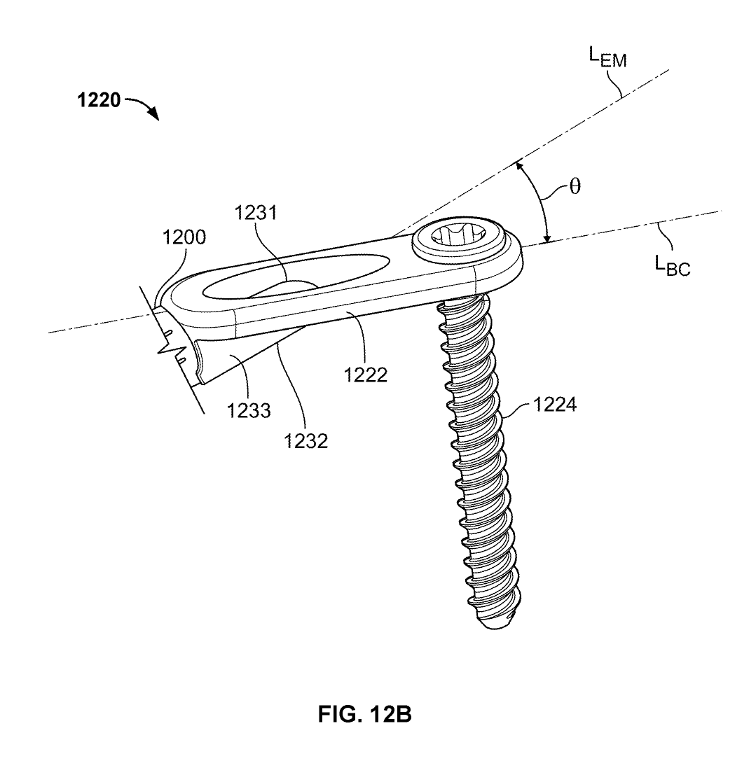

FIG. 13 shows a view taken along lines 13-13 (shown in FIG. 12) of implant 1200 in bone B. Anchor 1302 penetrates web 1202 at cell 1304. Anchor 1302 exits web 1202 at cell 1306. Engagement of web 1202 at two different cells may provide additional stability to anchor 1302. Engagement of web 1202 at two different cells may enable moment to be transmitted between web 1202 and anchor 1302. Anchor 1308 may also enter through one cell, traverse across the inside of web 1202 and exit web 1202 at a different cell.

Web 1202 includes cells that face in directions radially about the length of implant 1200 such that anchors 1302 and 1308 may be placed at a range of angles relative to each other.

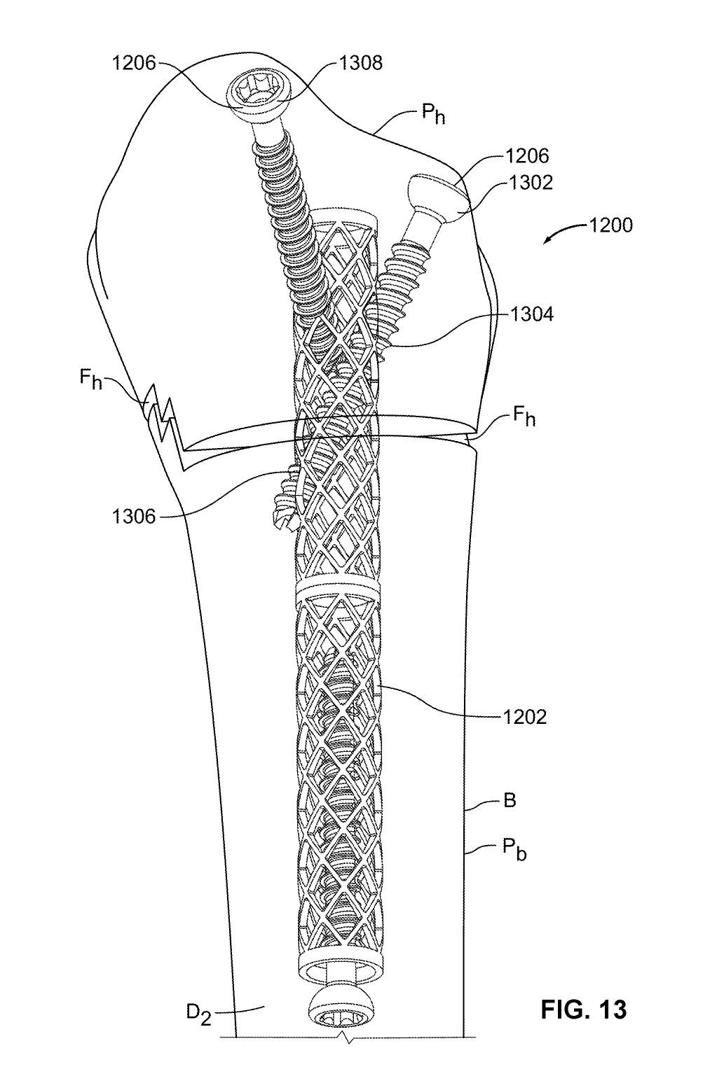

FIG. 14 shows implant 1400. Implant 1400 may include solid tubular portion 1402. Implant 1400 may include webbed portion 1404. Implant 1400 may include saw portion 1406.

Distal end 1408 of implant 1400 may be engaged by a rotation source such as a drill handle (not shown) to rotate implant 1400 about its longitudinal axis. The rotation source may include a manual handle. The rotation source may include a power drill motor. When rotating, teeth 1410 may cut into a bone such as B (shown in FIG. 1) to provide an access hole that leads to the interior of bone B.

Webbed portion 1404 may be deployed in the interior. Solid tubular portion 1402 may be deployed in the interior. Anchor receiving cells 1412 may receive anchors that secure bone fragments such as one or more of P_{B}, Pₐ and Pₕ to implant 1400.

Implant 1400 may be deployed in any suitable position in bone B. For example, implant 1400 may span from subchondral position S1 to subchondral position S₂. Implant 1400 may span from one of the subchondral positions to one of diaphyseal position D₁ and diaphyseal position D₂. Implant 1400 may span from one of the diaphyseal positions to another of the diaphyseal positions.

Implant 1400 may be used as one or more the elements of truss 100 (shown in FIG. 1). Implant 1400 may be used as one or more of the elements of implant 100 (shown in FIG. 10).

Distal end 1408 may include a scoop (not shown). The scoop may have one or more features in common with scoop 110 (shown in FIG. 1).

Implant 1400 may have overall length Lₚ. Solid tubular portion 1402 may have length Lₜ. Webbed portion 1404 may have length L. Lengths Lₜ and Lₓ may have any suitable magnitude relative to length Lₚ. Solid tubular portion 1402 and webbed portion 1404 may each occupy any suitable position along length Lₚ. Solid tubular portion 1402 and webbed portion 1404 may be present in any suitable order relative to each other.

Implant 1400 may include more than one solid tubular portion such as solid tubular portion 1400. Implant 1400 may include more than one webbed portion such as webbed portion 1404.

Circumferential teeth 1414 may retain a plug of bone B. The plug may be removed after cutting the access hole. The plug may be left inside implant 1400 to promote healing. Tissues other than the plug may be cored by, or retained inside, implant 1400 and left inside implant 1400 to promote healing.

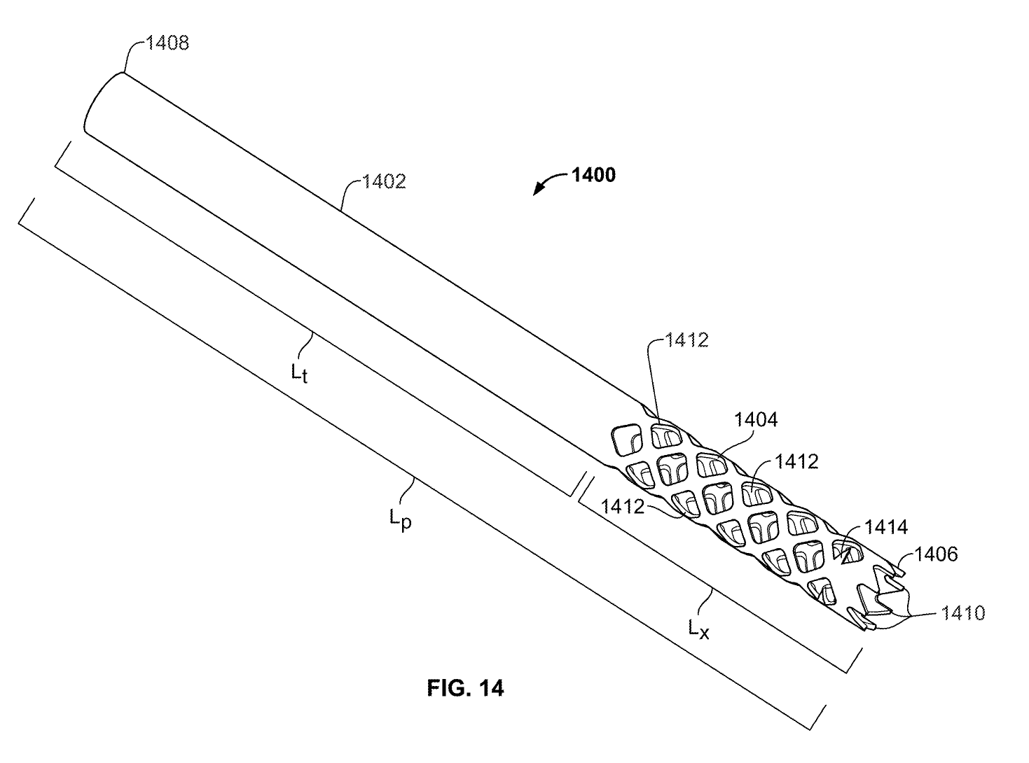

FIG. 15 shows illustrative double web 1500. Double web 1500 may include outer web 1502. Double web 1500 may include inner web 1504. Double web 1500 may be included in tubular implants such as implant 900 (shown in FIG. 9), implant 1000 (shown in FIG. 10), implant 1200 (shown in FIG. 12), implant 1400 (shown in FIG. 14) and any other suitable implants.

Outer web 1502 may be expandable. Inner web 1504 may be expandable.

Outer web 1502 and inner web 1504 may include anchor receiving cells such as 1506 and 1508, respectively. Cells 1506 may have a uniform cell density along the length of web 1502. Cells 1506 may have a cell density that varies along the length of web 1502. Cells 1508 may have a uniform cell density along the length of web 1504. Cells 1508 may have a cell density that varies along the length of web 1504. Cell density along web 1502 may be the same as or different from cell density along web 1504.

An anchor (not shown) that penetrates web 1502 may also penetrate web 1504. The anchor may engage web 1502 at an entry cell and at an exit cell. The anchor may engage web 1504 at an entry cell and at an exit cell. An anchor may thus engage double web 1500 at 1, 2, 3 or 4 cells. As the number of engagements increases, the strength of fixation of the anchor to double web 1500 increases. As the distances between the engagements increases, the strength of fixation of the anchor to double web 1500 increases.

Outer web 1502 and inner web 1504 may be held in a substantially coaxial configuration by bushings, hubs, collars or any other suitable mechanisms.

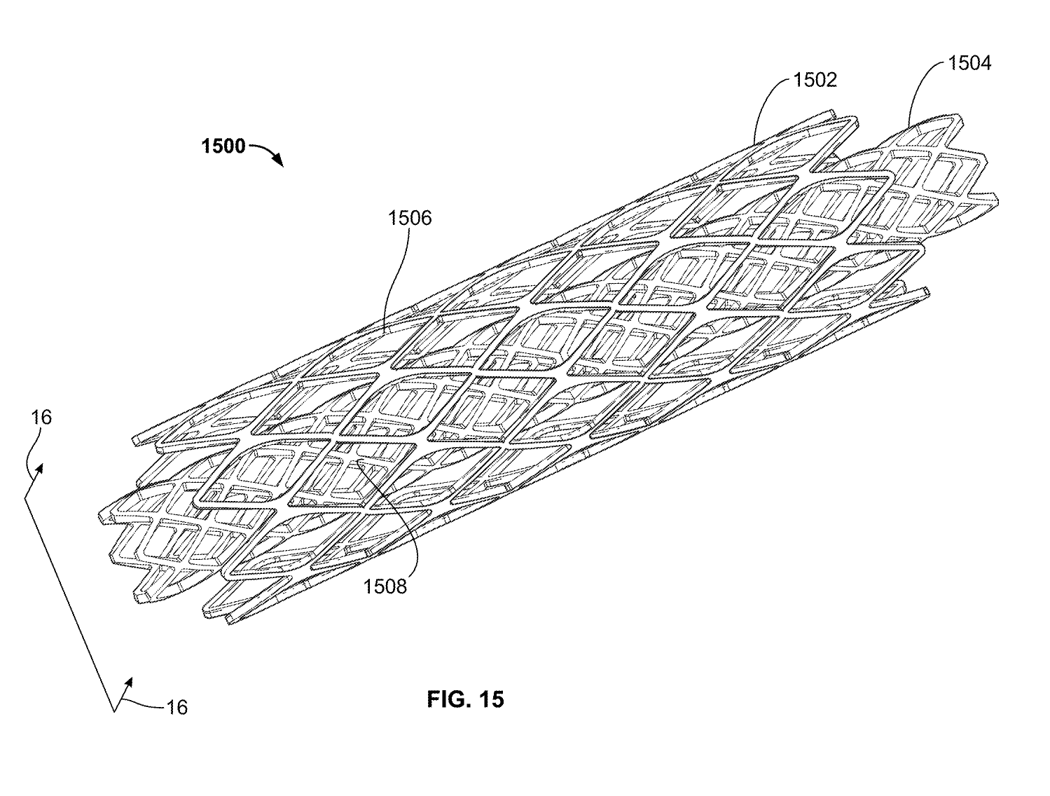

FIG. 16 shows a view of double web 1500 taken along lines 16-16 (shown in FIG. 15).

Some embodiments may include an implant that includes inner web 1504. Inner web 1504 may be expandable. When inner web 1504 is in an expanded state, it may have a greater diameter than when it is in a contracted state. The view shown in FIG. 16 shows diameters D_{c} and Dₑ, which may correspond to the contracted and expanded diameters, respectively.

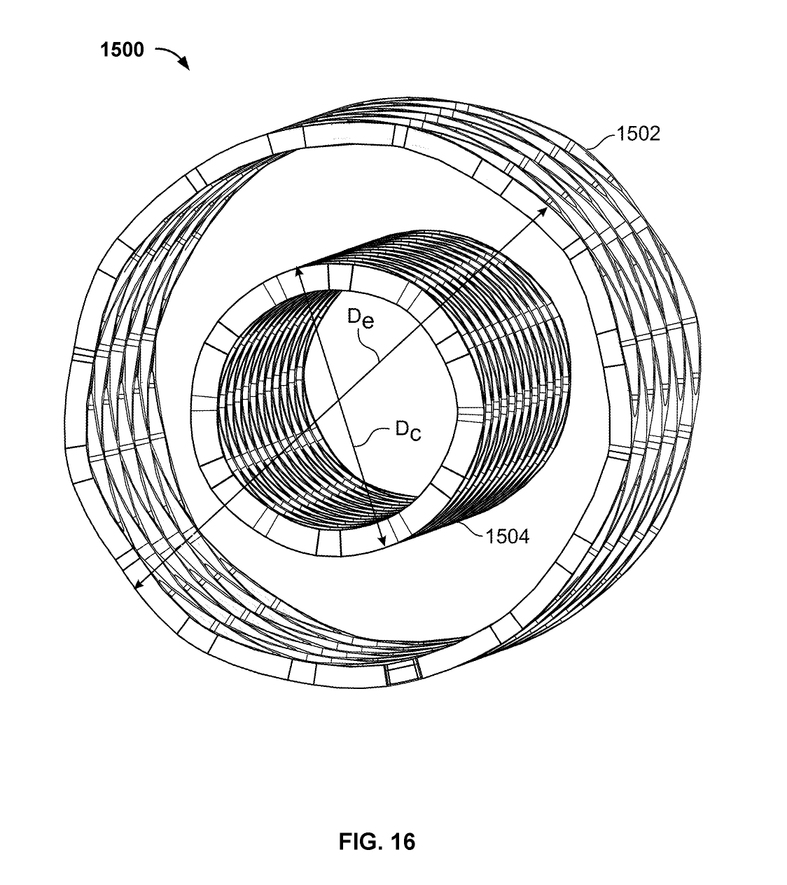

FIG. 17 shows illustrative implant 1700 in bone B. In FIG. 17, bone B is illustrated as including two fragments: P_{b} and Pₕ, which are separated by fracture Fₕ. Implant 1700 or portions thereof may be used in connection with two-part fractures, three-part fractures or fracture having more than three parts.

Implant 1700 may include web 1704. Web 1704 may include one or more anchor receiving features.

Implant 1700 may include an additional web or additional webs. The additional web or webs may be internal or external to web 1704. The additional web or webs may provide additional anchor engagement features. The additional engagement features may provide additional strength to an engagement of an anchor with implant 1700.

The anchor receiving features may include cells such as cell 1702. Anchors such as anchors 1706 may secure fragments Pₕ and P_{b} to implant 1700.

Implant 1700 may span across longitudinal bisecting plane P_{lb} (shown in FIG. 1) from subchondral position S₂ to subchondral position S₁.

Diagonal anchor 1708 may engage web 1704 of implant 1700. Diagonal anchor 1708 may engage cortical bone at diaphyseal position D₂. Diagonal anchor 1708 may span across longitudinal bisecting plane P_{lb} from diaphyseal position D₂ to web 1704. Diagonal anchor 1708 may not span across longitudinal bisecting plane P_{lb} from diaphyseal position D₂ to web 1704.

When one or more additional webs are present in implant 1700, anchor 1708 may engage the one or more additional webs.

Diagonal anchor 1708 may be used to transmit load from an end bone fragment such as Pₕ to a long bone fragment such as P_{b}.

Implant 1700 may be delivered to the interior of bone B in a manner that is analogous to the delivery of subchondral member 102 and diaphyseal member 134 (shown in FIG. 1).

Implant 1700 may include central axis member 1710. Implant 1700 may include proximal base 1712. Implant 1700 may include distal base 1714. Displacement of proximal base 1712 axially away from distal base 1714 may cause web 1704 to collapse toward central axis member 1710. Displacement of proximal base 1712 axially toward distal base 1714 may cause web 1704 to expand away from central axis member 1710.

At a particular axial position on web 1704, web 1704 may have a density of cells around the circumference of web 1704. The density of cells may be different for different axial positions on web 1704. In this way, web 1704 may have an expanded radius that varies axially on web 1704. Implant 1700 may thus have a shape that is defined by the cell density along web 1704. The shape may be non-cylindrical.

Any suitable broach may be used to shape a cavity inside bone B to conform to a non-cylindrical shape of implant 1700.

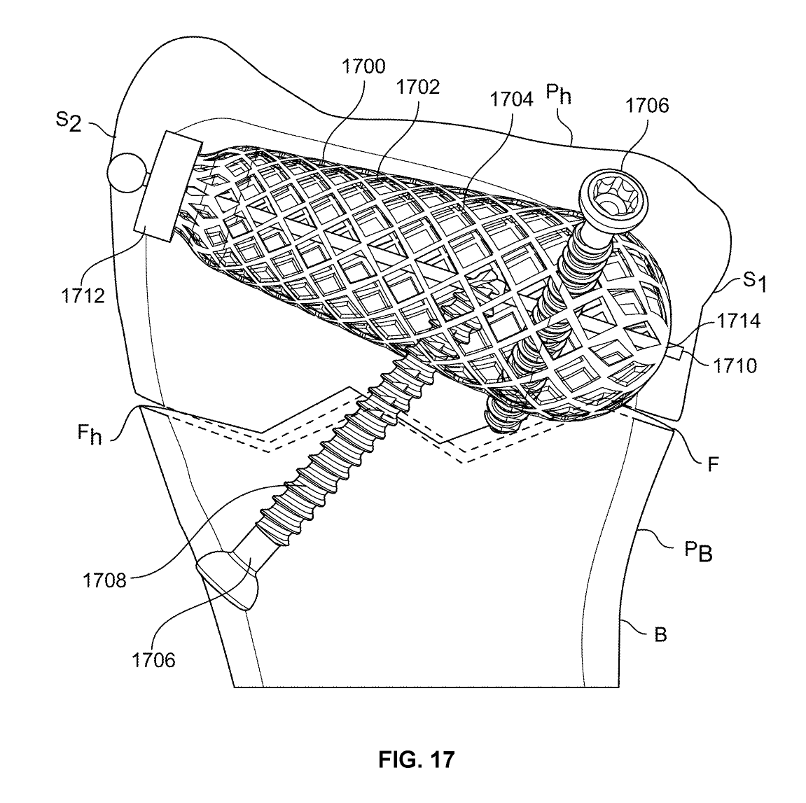

FIG. 18 shows illustrative instrument guide 1800 positioned at site H′ on bone B. H′ is illustrated as being a diaphyseal position, but H′ could also be a subchondral position for broaching an access hole such as Hₛ (shown in FIG. 5).

Broach head 1824 may be resilient such that broach head displaces cancellous bone B_{CA}, but not cortical bone B_{CO}, even at a fracture, where sharp cortical bone protrusions may be present. Broach head 1824 may be delivered through guide 1800 to target region Rₜ of intramedullary space IS. Target region Rₜ is illustrated as being within cancellous bone B_{CA}, but could be in either, or both, of cancellous bone B_{CA} and cortical bone B_{CO}. Side template 1830 and top template 1832 are registered to guide tube 1820. Arm 1831 may support template 1830. A practitioner may position templates 1830 and 1832 such that templates 1830 and 1832 “project” onto target region Rₜ so that guide 1800 will guide broach head 1824 to target region Rₜ.

Template 1830 may include lobe outline 1834 and shaft outline 1836 for projecting, respectively, a “swept-out” area of broach head 1824 and a location of shaft-like structure 1825. Template 1832 may include lobe outline 1838 and shaft outline 1840 for projecting, respectively, a target “swept-out” area of broach head 1824 and a target location of shaft-like structure 1825. Templates 1830 and 1832 may be configured to project a shape of any suitable instrument that may be deployed, such as a drill, a coring saw, a prosthetic device or any other suitable instrument.

Fluoroscopic imaging may be used to position templates 1830 and 1832 relative to target region Rₜ.

Broach head 1824 may rotate in intramedullary space IS to clear intramedullary bone matter so that a prosthetic device may be implanted. Broach head 1824 may be driven and supported by broach control 1826 and broach sheath 1827.

Guide 1800 may include base 1802. Alignment members 1804 and 1806 may extend from base 1802 to align guide centerline CL_{G} of guide 1800 with bone centerline CL_{BS} of the top surface of bone B. One or both of alignment members 1804 and 1806 may be resilient. One or both of alignment members 1804 and 1806 may be stiff.

Alignment members 1804 and 1806 may be relatively free to slide along surfaces of bone B. Guide 1800 may include contacts 1808 and 1810 that may engage bone B along centerline CL_{BS}. Contacts 1808 and 1810 may extend from a bottom surface of guide 1800. Contacts 1808 and 1810 may prevent guide centerline CL_{G} from rotating out of alignment with bone centerline CL_{BS}.

Contacts 1808 and 1810 may assure alignment of guide 1800 with the surface of bone B, because two points of contact may be stable on an uneven surface even in circumstances in which 3, 4 or more contacts are not stable.

Guide 1800 may include lateral cleats 1812 and 1814. Lateral cleats 1812 and 1814 may engage the surface of bone B to prevent guide 1800 from rotating in direction θ about guide centerline CL_{G}. Lateral cleats 1812 and 1814 may be resilient to allow some sliding over bone B.

When a practitioner positions guide 1800 on bone B, alignment members 1804 and 1806 may be the first components of guide 1800 to engage bone B. Alignment members 1804 and 1806 may bring guide centerline CL_{G} into alignment with bone centerline CL_{BS} before contacts 1808 and 1810 and cleats 1812 and 1814 engage bone B. Then, in some embodiments, cleats 1812 and 1814 may engage bone B to inhibit rotation in direction θ. Then, in some embodiments, contacts 1808 and 1810 may engage bone B along bone centerline CL_{BS}. Contacts 1808 and 1810 may have sharp points to provide further resistance to de-alignment of guide centerline CL_{G} from bone centerline CL_{BS}. In some embodiments, there may be no more than two contacts (e.g., 1808 and 1810) to ensure that the contacts are in line with bone centerline CL_{BS}.

Guide 1800 may include stem 1816 and grip 1818. A practitioner may manually grip 1818. In some embodiments, a torque-limiter (not shown) may be provided to limit the torque that the practitioner can apply via grip 1818 to contacts 1808 and 1810.

Guide tube 1820 may receive and guide any suitable instrument. Guide tube 1820 may be oriented at angle α with respect to handle 1816. In some embodiments, angle α may be fixed. In some embodiments, angle α may be adjustable. In some embodiments, templates 1830 and 1832 may be fixed relative to guide tube 1820. In some embodiments, including some embodiments in which α is adjustable and some in which α is not adjustable, guide tube 1820 may be oriented so that the axis L_{GT} of guide tube 1820 intersects bone B at substantially the same point as does axis L_{H} of stem 1816. Grip 1818 will thus be positioned directly over the center of hole site H′.

Guide 1800 may include channels 1842 and 1844. Rods 1846 and 1848 may be inserted through channels 1842 and 1844, respectively, through cortical bone B_{CO}. Rods 1846 and 1848 may stabilize guide 1800 on bone B. Rods 1846 and 1848 may be K-wires. Rods 1846 and 1848 may be inserted using a wire drill.

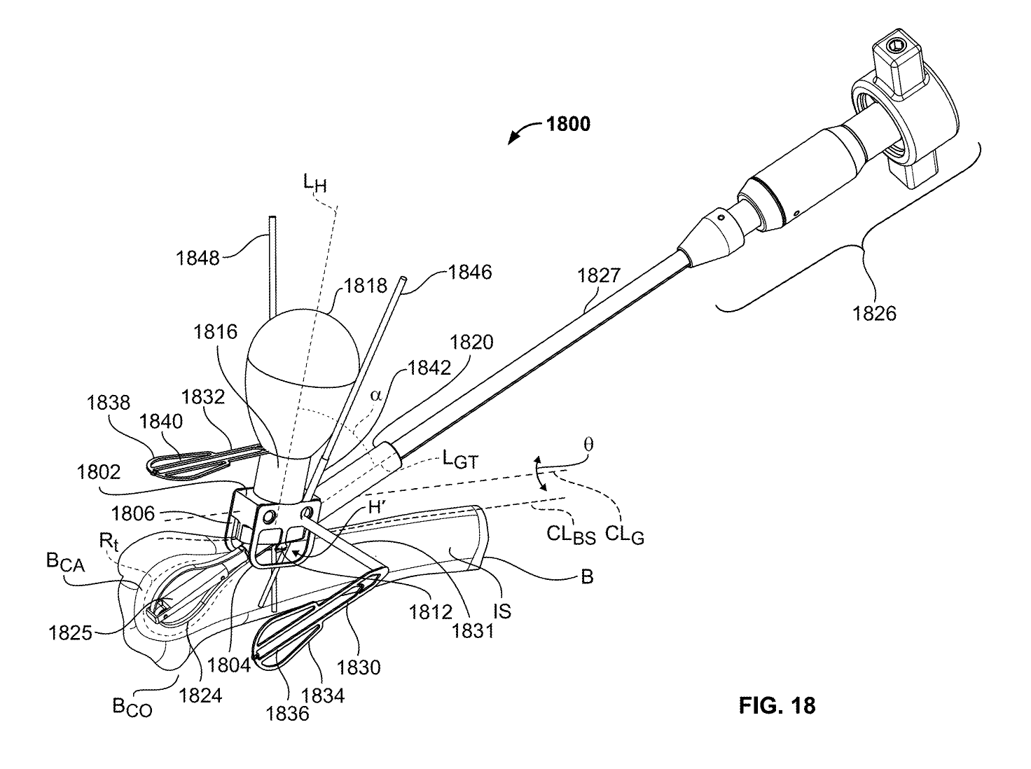

FIG. 19 shows illustrative web 1900. Web 1900 may be representative of webs that may be used in connection with implants shown and described herein. For example, a web such as web 1900 may be included in implant 900 (shown in FIG. 9), implant 1000 (shown in FIG. 10), implant 1200 (shown in FIG. 12), implant 1400 (shown in FIG. 14), implant 1700 (shown in FIG. 17) and any other suitable implants.

Web 1900 may include one or more cells such as cell 1902. Cell 1902 is configured to receive anchor 1904. Anchor 1904 may have one or more features in common with anchors such as anchors 106, 116 and 134 (shown in FIG. 1), 1006, 1016 and 1020 (shown in FIG. 10), 1206 and 1208 (shown in FIG. 12) and any other suitable anchors.

Cell 1902 may have an opening that is large enough to allow passage of anchor root 1906 through cell 1902 without deformation of cell 1902 when anchor 1904 is oriented normal to cell 1902. Such a cell may be referred to as an “open cell.” If anchor 1904 were to penetrate cell 1902 at an oblique angle, such that less than the full opening of cell 1902 were present in a plane normal to anchor 1904, cell 1902 may deform to accommodate root 1906.

Cell 1902 may be open by virtue of expansion from a closed state. Cell 1902 may be fabricated in an open state. Cell 1902 may be implanted in bone B (shown in FIG. 2) in an open state. Cell 1902 may be implanted in bone B (shown in FIG. 2) in a closed state. Cell 1902 may be expanded after deployment in bone B.

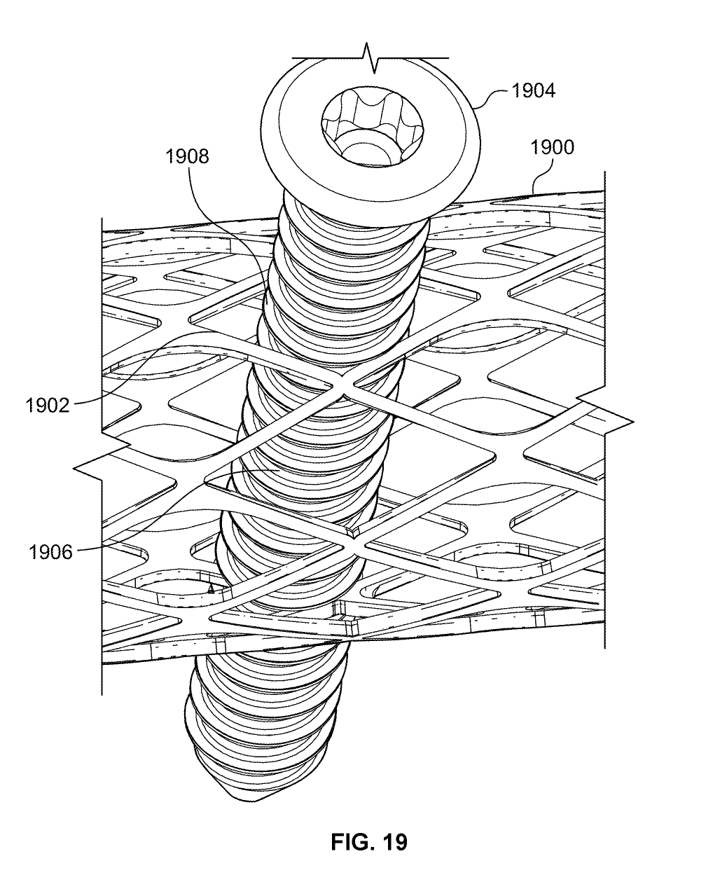

FIG. 20 shows illustrative tubular web 2000. (Web 2000 may be cylindrical about axis L_{w}. Only a portion of web 2000 in the foreground of axis L_{w} is shown.) Web 2000 may be representative of webs that may be used in connection with implants shown and described herein. For example, a web such as web 2000 may be included in implant 900 (shown in FIG. 9), implant 1000 (shown in FIG. 10), implant 1200 (shown in FIG. 12), implant 1400 (shown in FIG. 14), implant 1700 (shown in FIG. 17) and any other suitable implants.

Web 2000 may include one or more cells such as cell 2002. Cell 2002 is configured to receive anchor such as 1904 (shown in FIG. 19).

Cell 2002 may have an opening that is not large enough to allow passage of anchor root 1906 through cell 2002 without deformation of cell 2002 when anchor 2004 is oriented normal to cell 2002. Such a cell may be referred to as a “closed cell.” If anchor 2004 were to penetrate cell 2002 at a normal angle, such that the full opening of cell 2002 were present in a plane normal to anchor 1904, cell 2002 would have to deform to accommodate root 2006.

Cell 2002 may have a mechanical equilibrium state in which cell 2002 is closed. Cell 2002 may be deployed in bone B (shown in FIG. 2) in the closed mechanical equilibrium state. Cell 2002 may be used to secure bone fragments by receiving an anchor. The anchor may be an anchor that has a root, but no anchor engaging features, such as a K-wire. Cell 2002 may have a mechanical equilibrium state in which cell 2002 is open.

Both open and closed cells may be engaged by anchors having roots oriented at a wide range of angles to the cell. Because close cells must deform to receive the root anchor, closed cells may require relatively more support from “behind” to engage an anchor.

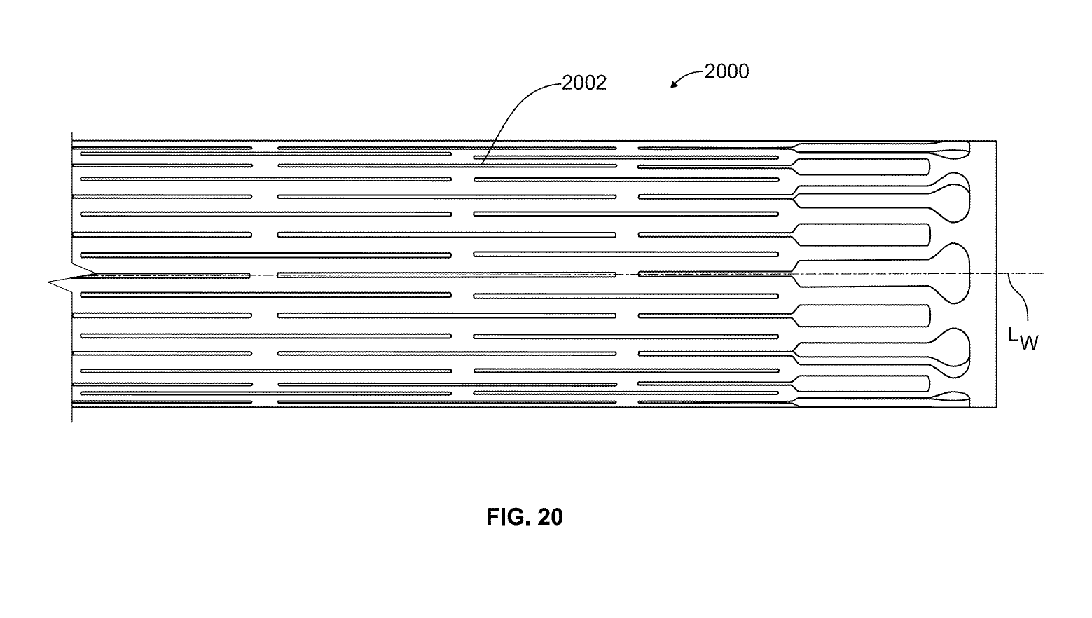

FIG. 21 shows illustrative guide 2100. Guide 2100 may be used to deploy one or more implants in bone B. The implants may be deployed in access holes such as one or more of the access holes shown in FIG. 5, access holes described herein in connection with implants, or any other suitable access holes. For example, guide 2100 may be used to deploy one or more implants such as one or more of the elements of truss 100 (shown in FIG. 1), implant 900 (shown in FIG. 9), implant 1000 (shown in FIG. 10), implant 1200 (shown in FIG. 12), implant 1400 (shown in FIG. 14), implant 1700 (shown in FIG. 17) and any other suitable implants.

For simplicity, fractures such as Fₕ and Fₐ (shown in FIG. 2) are not shown. Bone fragments such as P_{b}, Pₐ and Ph (shown in FIG. 2) may be provisionally reduced using K-wires before implantation of implants using guide 2100.

Guide 2100 may include articulating frame 2102. Frame 2102 may include reference arm 2104. Frame 2102 may include reference arm 2106. Reference arm 2104 may be hinged to reference arm 2106 at hinge 2107. Reference arm 2104 may support guide tube 2108. Reference arm 2104 may support guide tube 2109. Reference arm 2106 may support guide tube 2110. Reference arm 2106 may support guide tube 2112.

Guide 2100 may be configured to install elements E₁, E₂ and E₃ of an illustrative bone truss. Elements E₁, E₂ and E₃ may correspond to truss elements of a truss such as truss 100 (shown in FIG. 1). Elements E₁ and E₂ may intersect at joint J.

Reference arm 2104 may be registered to element E₁ by coaxially aligning guide tube 2108 with element E₁ and aligning guide tube 2109 with joint J₁.

An anchor such as A₁ may be deployed through guide tube 2109. An anchor such as A₂ may be deployed through a guide tube (not shown) that is supported at one of positions 2114. Each of positions 2114 may be registered to a corresponding one of anchor receiving features 2116.

Reference arm 2106 may be moved through angle δ to align guide tubes 2110 and 2112 for deployment of elements E₂ and E₃, respectively.

Element E₃ may be advanced to engage anchor receiving feature R₀. Anchor receiving feature R₀ may include one or more of anchor receiving feature 122, anchor receiving feature 700, anchor receiving feature 1008, anchor receiving feature 1032, anchor receiving feature 1203, cell 1902, cell 2002, anchor receiving feature 2116 and any other suitable anchor receiving feature.

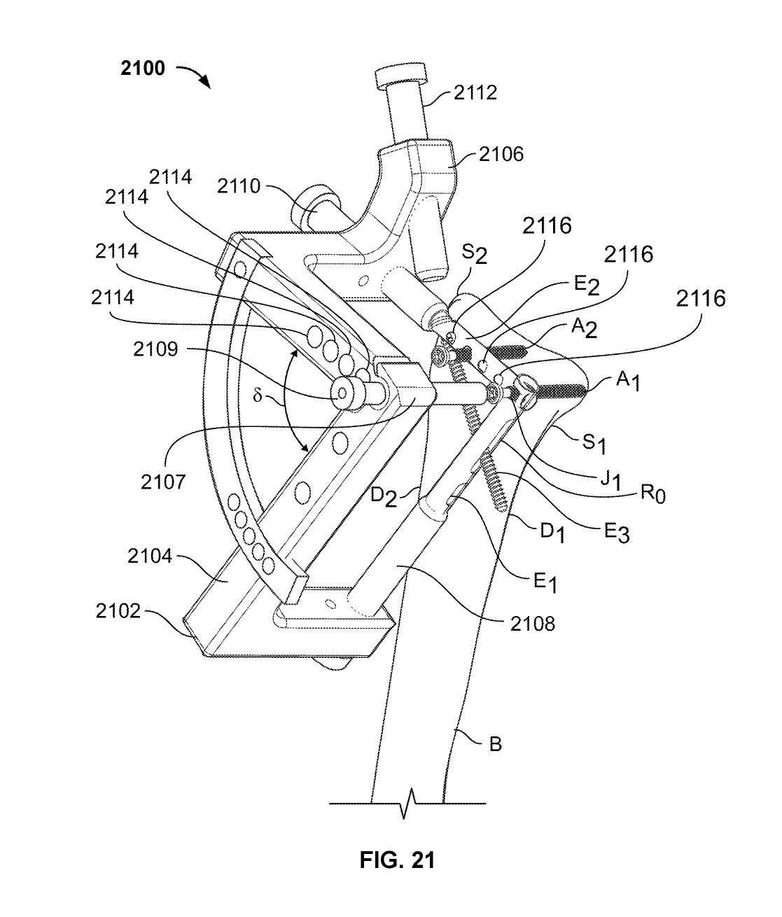

FIG. 22 shows illustrative guide 2200. Guide 2200 may be used to deploy one or more anchors in bone B. For example, guide 2200 may be used to deploy one or more anchors for an implant such as one or more of the elements of truss 100 (shown in FIG. 1), implant 900 (shown in FIG. 9), implant 1000 (shown in FIG. 10), implant 1200 (shown in FIG. 12), implant 1400 (shown in FIG. 14), implant 1700 (shown in FIG. 17) and any other suitable implants.

For simplicity, fractures such as Fₕ and Fₐ (shown in FIG. 2) are not shown. Bone fragments such as P_{b}, Pₐ and Pₕ (shown in FIG. 2) may be provisionally reduced using K-wires before implantation of implants using guide 2100.

A K-wire may be used to drill pilot holes through a bone fragment. The K-wire may be aligned with an anchor receiving feature such as anchor receiving feature R in element E₄. The K-wire may be passed through the anchor receiving feature. The K-wire may be passed through a portion of bone B that is distal (relative to the anchor) the anchor receiving feature. A cannulated anchor such as cannulated anchor A₃ may then be introduced along the K-wire into the pilot hole. Cannulated anchor A₃ may be advanced to engage the anchor receiving feature. Cannulated anchor A₃ may be advanced to engage the distal bone portion. Cannulated anchor A₃ may thus be deployed to secure one or more bone portions to each other. Cannulated anchor A₃ may thus be deployed to secure one or more bone portions to element E₄.

Guide 2200 may include base 2202. Base 2202 may support pin 2204. Pin 2204 may engage element E₄ coaxially. Base 2202 may support rails 2206. Slidable guide 2208 may be slidable up and down rails 2206. End support 2212 may support rails 2206 opposite base 2202. Guide hole 2210 may be present in slidable guide 2208. Base 2202, pin 2204 and rails 2206 may be configured such that guide hole aligns with anchor receiving feature R₁.

Anchor receiving feature R₁ may include one or more of anchor receiving feature 122, anchor receiving feature 700, anchor receiving feature 1008, anchor receiving feature 1032, anchor receiving feature 1203, cell 1902, cell 2002, anchor receiving feature 2116 and any other suitable anchor receiving feature.

Guide hole 2210 may have one or more of an orientation, a length, a width and a diameter that is selected, based on the relative positions of base 2202, pin 2204 and rails 2206, to constrain tip T of K-wire K₁ to intersect anchor receiving feature R1 when K-wire K₁ advances through bone B. Slidable guide 2208 may be movable along rails 2206 to accommodate different sizes of element E4 and different locations of anchor receiving feature R₁ along the length of element E₄.

Base 2202 may pivot relative to pin 2204 while maintaining slidable guide 2208 at a fixed radius away from, and facing, element E₄. Base 2202 may pivot relative to pin 2204 while maintaining slidable guide 2208 at a fixed radius away from, and facing, anchoring feature R.

Base 2202 may include one or more pin receptacles 2214. Pin 2204 may be placed in an appropriate one of receptacles 2214 based on factors such as the angle of element E₄ relative to the long axis of bone B and other suitable factors, soft tissue thicknesses, clearance of associated equipment, and other operational considerations.

One or more of receptacles 2214 may be used to support an auxiliary alignment arm (not shown), a bushing support (not shown), or other auxiliary equipment.

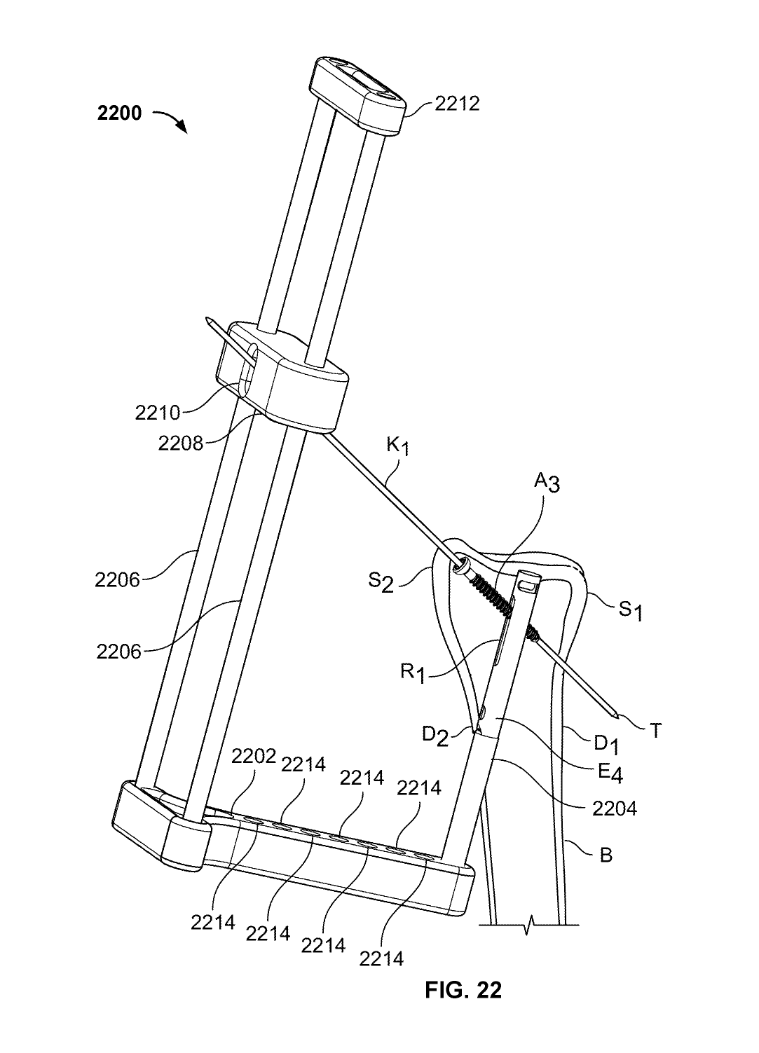

FIG. 23 shows illustrative guide 2300. Guide 2300 may be used to deploy one or more anchors in bone B (shown in FIG. 2). The anchors may be K-wires, screws or any other suitable anchors. For example, guide 2300 may be used to deploy one or more anchors for an implant such as one or more of the elements of truss 100 (shown in FIG. 1), implant 900 (shown in FIG. 9), implant 1000 (shown in FIG. 10), implant 1200 (shown in FIG. 12), implant 1400 (shown in FIG. 14), implant 1700 (shown in FIG. 17) and any other suitable implants.

Guide 2300 may include one or more bases such as base 2302. Base 2302 may include receptacle 2304 for supporting an implant such as implant E₅ perpendicular to base 2302. Implant E₅ is illustrated as a coring implant. Implant E₅ may include coring teeth C. Implant E₅ may include anchor receiving feature R₂. Implant Eₛ may include anchor receiving feature R₃. Implant E₅ may include any suitable number and any suitable type of anchor receiving features. For example, anchor receiving features R₂ and R₃ may include one or more of anchor receiving feature 122, anchor receiving feature 700, anchor receiving feature 1008, anchor receiving feature 1032, anchor receiving feature 1203, cell 1902, cell 2002, anchor receiving feature 2116 and any other suitable anchor receiving feature.

Base 2302 may support reference arm 2306 parallel to the direction in which implant E₅ is to be supported.

Reference arm 2308 may include guide hole 2308.

Base 2302, receptacle 2304 and reference arm 2306 may be configured such that guide hole 2308 aligns with one or more of anchor receiving features R₂ and R₃.

Guide hole 2308 may have one or more of an orientation, a length, a width and a diameter that is selected, based on the relative positions of base 2302, receptacle 2304 and reference arm 2306, to constrain tips T₂ and T₃ of K-wires K₂ and K₃ to align with the longitudinal axis of implant E₅ to facilitate intersection of the K-wires with anchor engaging features R₂ and R₃.

Base 2302 may pivot relative to implant E₅ while maintaining reference arm 2306 at a fixed radius away from, and facing, element E₅. Base 2302 may pivot relative to implant E₅ while maintaining reference arm 2306 at a fixed radius away from, and facing, anchoring features R₂ and R₃.

Elements 2302′ and 2302″ may represent alternative circumferential positions of base 2302 relative to anchor receiving features R₂ and R₃. For example, element 2302′ is shown at an angle η circumferentially away from base 2302. Alternatively, elements 2302′ and 2302″ may represent embodiments of guide 2300 that include one, two or more than two bases. In those embodiments, bases 2302, 2302′, 2302″ may share receptacle 2304. In some embodiments, one or more of bases 2302, 2302′ and 2302″ may be circumferentially fixed relative to another of the bases. In some embodiments, one or more of bases 2302, 2302′ and 2302″ may be hinged and circumferentially displaceable relative to another of the bases.

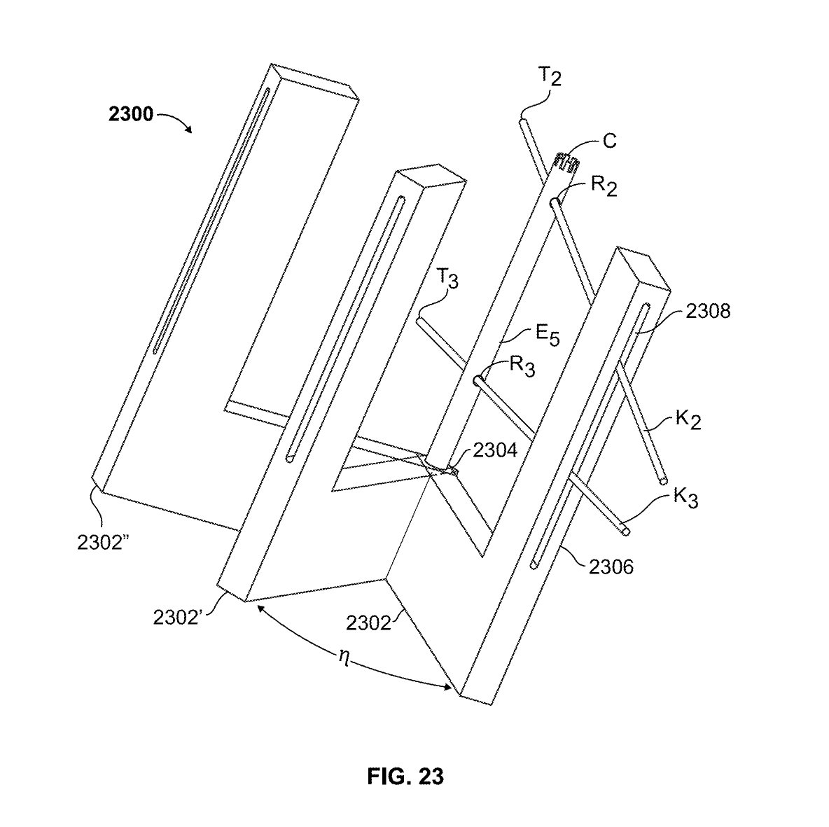

FIG. 24 shows illustrative multi-element implant 2400. Implant 2400 may include two or more elongated elements. Implant 2400 is illustrated as including 5 elements: 2402, 2404, 2406, 2408 and 2410. One, some or all of elements 2402, 2404, 2406, 2408 and 2410 may have features in common with elements of implants such as truss 100 (shown in FIG. 1), implant 900 (shown in FIG. 9), implant 1000 (shown in FIG. 10), implant 1200 (shown in FIG. 12), implant 1400 (shown in FIG. 14), implant 1700 (shown in FIG. 17) and any other suitable implants. For example, one, some or all of elements 2402, 2404, 2406, 2408 and 2410 may include a web of anchor receiving cells. One, some or all of elements 2402, 2404, 2406, 2408 and 2410 may be expandable. One, some or all of elements 2402, 2404, 2406, 2408 and 2410 may not be expandable.

Elements 2402, 2404, 2406, 2408 and 2410 may each contribute structural strength to implant 2400. Elements 2402, 2404, 2406, 2408 and 2410 may each contribute anchor receiving features to implant 2400. In embodiments of implant 2400 in which elements 2402, 2404, 2406, 2408 and 2410 include webs of anchor receiving cells, an anchor such as 1904 (shown in FIG. 19) may engage 1, 2, 3, 4 or more cells along a linear path. As the number of engaged cells increases, the ability of implant 2400 to transmit tensile stress axially along the anchor and bending moment perpendicularly to the axis of the anchor increases.

Implant 2400 may include retainer 2410. Retainer 2410 may maintain proximity of elements 2402, 2404, 2406, 2408 and 2410. Retainer 2410 may include rings 2412. Rings 2412 may seat adjacent stress relief cuts 2414 in elements 2402, 2404, 2406, 2408 and 2410. Plugs 2416 may be seated in the ends of elements 2402, 2404, 2406, 2408 and 2410 to expand the ends and retain rings 2412 in position. Rings 2412 may be fixed relative to each other to retain the ends of elements 2402, 2404, 2406, 2408 and 2410.

Implant 2400 may include retainer 2418. Retainer 2418 may maintain proximity of elements 2402, 2404, 2406, 2408 and 2410 at location spaced apart longitudinally from retainer 2410.

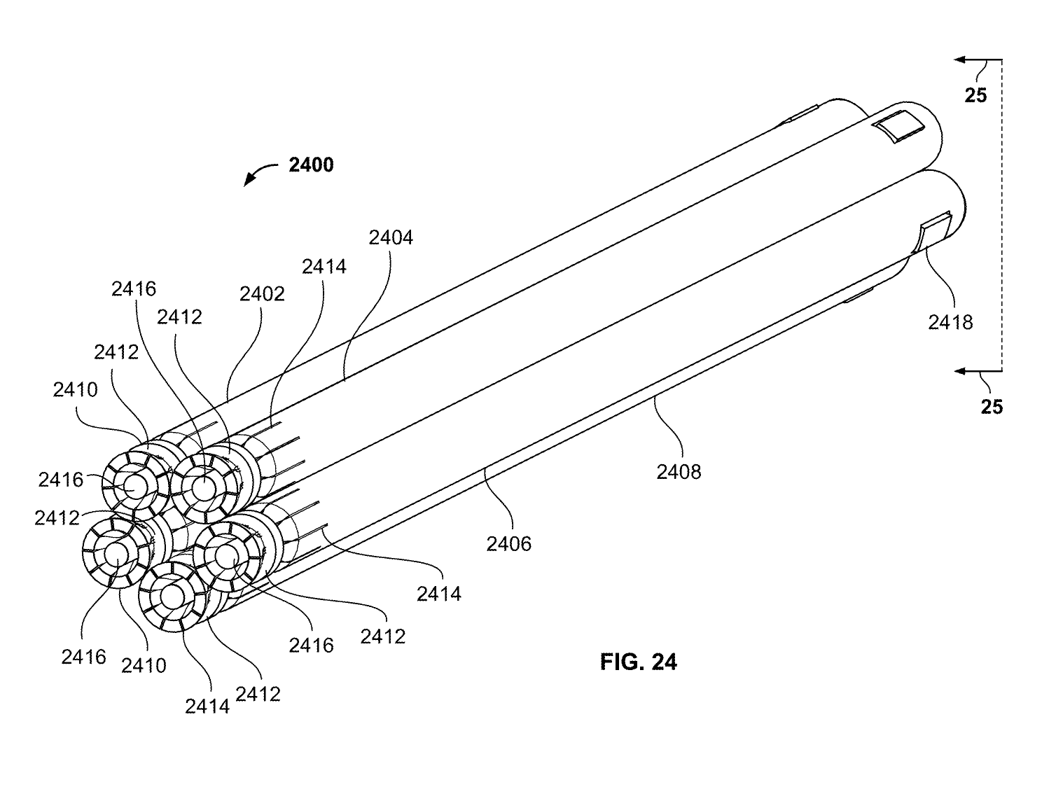

FIG. 25 shows a view of implant 2400 taken along lines 25-25 (shown in FIG. 24). Retainer 2418 may be positioned along longitudinal axis L_{M} of implant 2400. Retainer 2418 may include radial arms 2420 that extend along radius R and pass through radially inner slots 2422 and radially outer slots 2424 in elements 2402, 2404, 2406, 2408 and 2410.

Radial arms 2420 may include detents (not shown) adjacent radially inner slots 2422 that retain portions of elements 2402, 2404, 2406, 2408 and 2410 at maximum radial positions. Radial arms 2420 may include detents (not shown) adjacent radially outer slots 2424 that retain portions of elements 2402, 2404, 2406, 2408 and 2410 at maximum radial positions. A radial arm 2420 for an expandable element may include a detent that corresponds only to a radially inner slot 2422 to allow radially outward portions of the element to displace away from axis L_{M} during expansion. The detent corresponding to inner slot 2422 may be radially outwardly displaced from the slot, when the element is collapsed, to accommodate expansion of the elements.

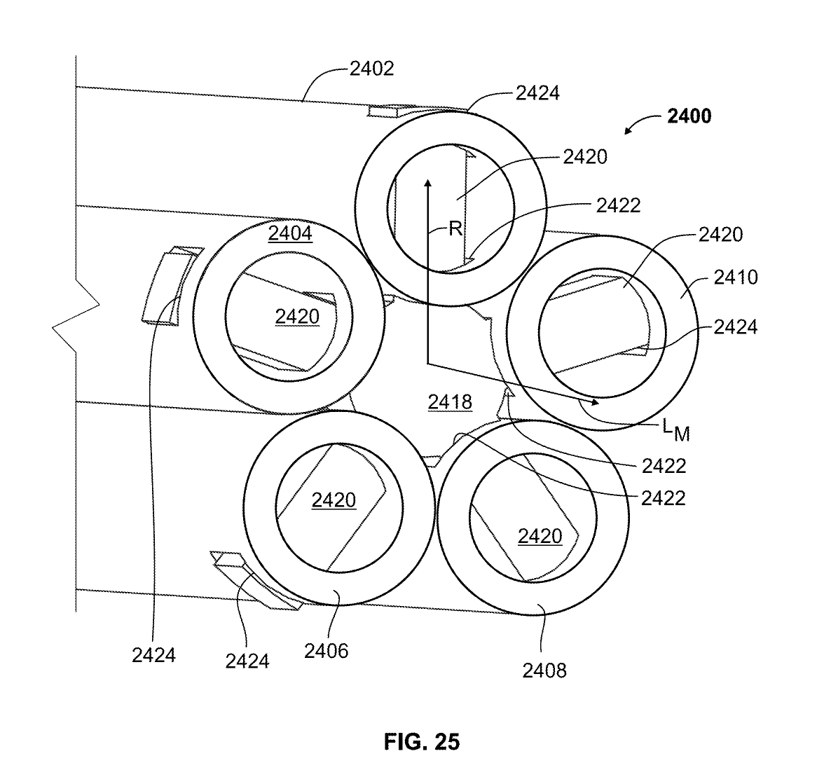

FIG. 26 shows an embodiment of implant 2500 in which elements 2402, 2404, 2406, 2408 and 2410 are expandable inside bone B. For the purpose of illustrating the expanded state of implant 2500, implant 2500 is shown without retainers 2410 and 2418. Elements 2402, 2404, 2406, 2408 and 2410 may include expandable webs such as web 1704. The webs may include anchor receiving cells (not shown) that vary in density along axis L_{M} such that at proximal end 2602, the overall diameter of implant 2500 is not as great as that at distal end 2604. The variation of cell density along longitudinal axis L_{M} may be the same for two or more of elements 2402, 2404, 2406, 2408 and 2410. The variation of cell density along longitudinal axis L_{M} may be different among two or more of elements 2402, 2404, 2406, 2408 and 2410.

Implants shown and described herein, such as truss 100 (shown in FIG. 1), implant 900 (shown in FIG. 9), implant 1000 (shown in FIG. 10), implant 1200 (shown in FIG. 12), implant 1400 (shown in FIG. 14), implant 1700 (shown in FIG. 17) and any other implants shown and described herein, may be used in any bone such as bone B (shown in FIG. 5). Table 2 includes a partial list of bones Sᵢ that may correspond to bone B. Bone B may correspond to any long bone.

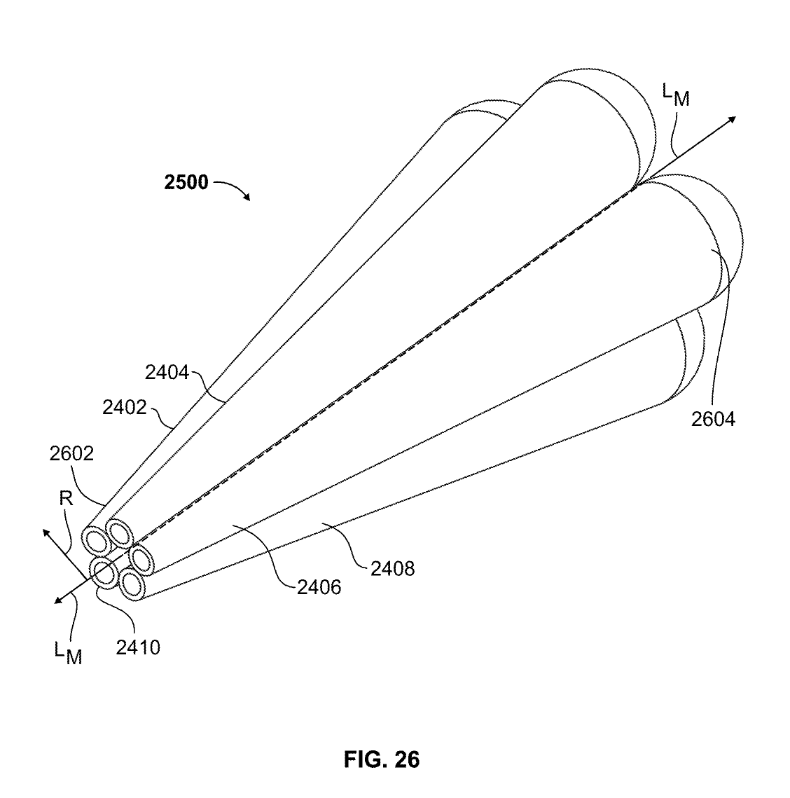

TABLE 2
Bones Sᵢ.

| | Reference |
|---|---|
| | numeral in |
| Bone | FIG. 27 |
| Distal Radius | S₀ |
| Humerus | S₁ |
| Proximal Radius and Ulna (Elbow) | S₂ |
| Metacarpals | S₃ |
| Clavicle | S₄ |
| Ribs | S₅ |
| Vertebrae | S₆ |
| Ulna | S₇ |
| Hip | S₈ |
| Femur | S₉ |
| Tibia | S₁₀ |
| Fibula | S₁₁ |
| Metatarsals | S₁₂ |

FIG. 27 shows illustrative skeleton S. Skeleton S includes illustrative bones Sᵢ.

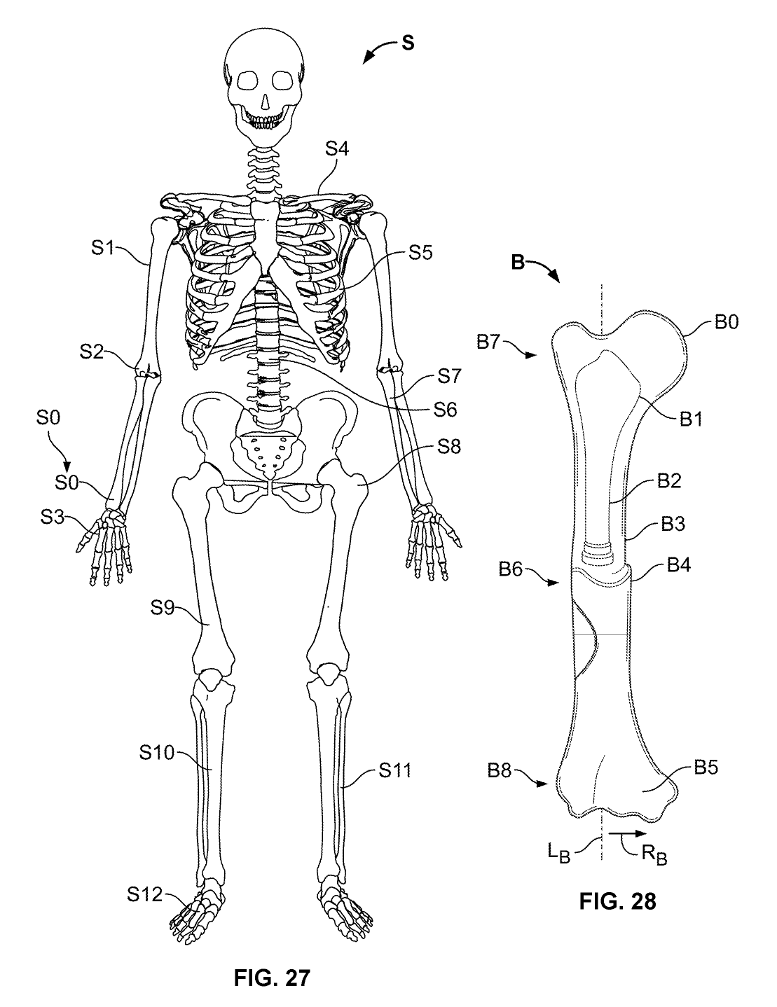

FIG. 28 schematically shows anatomy of bone B (shown in FIG. 5). Anatomical features of bone B are listed in Table 3. Apparatus and methods in accordance with the principles of the invention may involve one or more of the anatomical features shown in Table 3. Features of bone B may be described in reference to bone axis L_{B} (in which B indicates bone) and radius R_{B} (in which B indicates bone).

TABLE 3
Anatomical features of some of the bone types that
may be treated by the apparatus and methods.

| | Reference numeral |
|---|---|
| Anatomical feature | in FIG. 28 |
| Articular surface | B₀ |
| Cancellous, spongy or trabecular bone | B₁ |
| Medullary cavity | B₂ |
| Cortical or dense bone | B₃ |
| Periosteum | B₄ |
| Proximal articular surface | B₅ |
| Diaphysis or midshaft | B₆ |
| Metaphysis or end region | B₇ |
| Epiphysis | B₈ |
| Articular surface | B₉ |

The terms “end-bone” and “end-bone fracture” may refer to fractures that occur in the epiphyseal or metaphyseal region of long bones. Such fractures include peri-articular and intra-articular fractures.

Thus, apparatus and methods for fracture repair have been provided. Persons skilled in the art will appreciate that the present invention can be practiced by other than the described embodiments, which are presented for purposes of illustration rather than of limitation. The present invention is limited only by the claims that follow.

What is claimed is:

1. A bone truss comprising elongated members, each of the elongated members being configured to be:
inserted substantially fully into a bone; and, then,
locked to another of the elongated members, the elongated members defining a triangular region inside the bone.

2. The truss of claim 1 wherein the elongated members include a subchondral member.

3. The truss of claim 2 wherein:
the bone defines a bisecting longitudinal plane; and
the elongated members further include a first diagonal member that is configured to span from a first subchondral position to a second diaphyseal position that is diagonally across the plane from the first subchondral member.

4. The truss of claim 3 wherein the elongated members further include a second diagonal member that is configured to span from a second subchondral position to a first diaphyseal position that is diagonally across the plane from the second subchondral position.

5. The truss of claim 4 wherein the subchondral member is tubular.

6. The truss of claim 5 wherein the first diagonal member is tubular.

7. The truss of claim 4 wherein the elongated members further include a diaphyseal member that spans from the first diaphyseal position to the second diaphyseal position.

8. The truss of claim 7 wherein the subchondral member includes a subchondral tubular structure.

9. The truss of claim 8 wherein the subchondral tubular structure includes a cell that is one of a plurality of cells, each cell being configured to receive a bone anchor.

10. The truss of claim 9 wherein the cell is an open cell.

11. The truss of claim 10 wherein the cell is a closed cell.

12. The truss of claim 9 wherein the subcohondral tubular structure is expandable.

13. The truss of claim 8 wherein:
the first diagonal member includes a diagonal tubular structure; and
the diagonal tubular structure is configured to be joined at the first subchondral position directly to the subchondral tubular structure.

14. The truss of claim 8 wherein the diaphyseal member includes a diaphyseal tubular structure.

15. The truss of claim 14 wherein the diaphyseal tubular structure includes a cell that is one of a plurality of cells, each cell being configured to receive a bone anchor.

16. The truss of claim 15 wherein the cell is an open cell.

17. The truss of claim 16 wherein the cell is a closed cell.

18. The truss of claim 15 wherein the diaphyseal tubular structure is expandable.

19. The truss of claim 14 wherein the diaphyseal member is configured to be joined at the second diaphyseal position directly to the first diagonal member.

20. The truss of claim 14 wherein:
the second diagonal member is configured to transmit compressive force, in an outward radial direction relative to a longitudinal axis of the bone, to the first diaphyseal position; and
the diaphyseal member is configured to transmit tensile force, in an inward radial direction relative to the longitudinal axis, to the first diaphyseal position.

21. The truss of claim 20 wherein the second diagonal member and the diaphyseal member are configured such that the outward radial force has a magnitude that is approximately the same as a magnitude of the inward radial force.

22. The truss of claim 19 wherein:
the first diagonal member and the second diagonal member may be configured to form a node;
the first diagonal member is configured to transmit compressive force from the first subchondral position to the node; and
the node is configured to transmit:
a first portion of the compressive force along the first diagonal member to the second diaphyseal position; and
a second portion of the compressive force along the second diagonal member to the first diaphyseal position.

23. The truss of claim 19 wherein:
the first diagonal member and the second diagonal member may be configured to form a node;
the second diagonal member is configured to transmit compressive force from the first subchondral position to the node; and
the node is configured to transmit:
a first portion of the compressive force along the first diagonal member to the second diaphyseal position; and
a second portion of the compressive force along the second diagonal member to the first diaphyseal position.

24. A tubular implant for a bone, the tubular implant comprising:
a first end configured to couple subchondrally to the bone at a loading position; and
a second end configured to couple to the bone at a diaphyseal position that is across a longitudinally bisecting plane of the bone from the loading position.

25. The tubular implant of claim 24 wherein the second end terminates at a surface that is oblique to a length of the implant and substantially parallel to a diaphyseal surface of the bone.

26. The tubular implant of claim 25 further comprising an inner tubular surface, wherein the second end includes an anchor receiving feature in the inner tubular surface, the anchor receiving feature being configured to receive an anchor that is configured to penetrate cortical bone adjacent the anchor receiving feature and cortical bone that is across the longitudinally bisecting plane of the bone from the anchor receiving feature.

27. The tubular implant of claim 25 wherein, at the second end, the inner tubular surface defines a pocket that accommodates, between an inner wall of the cortical bone and an outer wall of the cortical bone, a portion of a head of the anchor.

28. The tubular implant of claim 24 further comprising a tubular wall that defines a first elongated window and a second elongated window opposite the first elongated window, each of the first and second elongated windows being configured to receive a body of an anchor and engage an engagement feature of the anchor.

29. The tubular implant of claim 28 wherein the first and second elongated windows are configured to cooperatively brace the anchor at an angle relative to the tubular implant, the angle being determined by an angle at which the anchor enters the first elongated window.

30. The tubular implant of claim 24 being expandable.

31. The tubular implant of claim 30 including a web of anchor receiving cells.

32. A method for treating an end of a bone, the method comprising:
preparing an elongated subchondral cavity that is transverse to a longitudinal axis of the bone;
expanding a web of anchor receiving cells in the subchondral cavity; and
engaging the web with an anchor that is anchored to a portion of the bone.

33. The method of claim 32 wherein the expanding includes expanding a web that has a central axis and a diameter that varies along the central axis.

34. An anchor receiving bone support comprising a tube wall that defines a first elongated window and a second elongated window opposite the first elongated window, each of the first and second elongated windows being configured to be traversed by a body of an anchor and engaged by an engagement feature of the anchor.

35. The support of claim 34 wherein the first and second elongated windows are configured to cooperatively brace the anchor at an angle relative to the tubular implant, the angle ranging from (a) perpendicular to the implant to (b) an angle that is defined by an outer diameter of the tubular implant, a radius of the anchor and a longitudinal displacement between an end of the first elongated window and an end of the second elongated window.

36. The support of claim 34 further comprising, when the tube wall is a first tube wall, a second tube wall having a transverse slot that is configured to be moved to different positions along the first and second elongated windows, the transverse slot being configured to be traversed by a body of the anchor and engaged by an engagement feature of the anchor.

37. The support of claim 36 wherein the first tube wall is nested inside the second tube wall.

38. The support of claim 36 wherein the second tube wall is nested inside the first tube wall.

39. The support of claim 36 wherein the first elongated window, the second elongated window and the transverse slot are configured to cooperatively brace the anchor against rotation relative to a longitudinal axis of the first tube wall.

40. A tubular bone support comprising:
a tubular web of anchor receiving cells; and
a ring of saw teeth configured to saw an access hole for delivering the bone support to a bone interior region.

41. The tubular bone support of claim 40 being configured to be locked into a bone support truss after being delivered to the interior region.

42. The tubular support of claim 40 further comprising a solid tube that is longitudinally contiguous with the web.

43. A bone anchor substrate comprising:
a first elongated member comprising a first web of anchor receiving features; and
a second elongated member comprising a second web of anchor receiving features;
wherein the second elongated member is configured to be deployed alongside the first elongated member in an interior region of a bone.

44. The bone anchor substrate of claim 43 wherein:
the first elongated member has a first delivery state diameter and is configured to be delivered to the interior region through a guide tube that has an inner diameter;
the second elongated member has a second delivery state diameter and is configured to be delivered to the interior region through the guide tube; and
a sum of the first and second delivery state diameters is greater than the inner diameter.

45. The bone anchor substrate of claim 43 wherein:
the first elongated member has a first delivery state diameter and is configured to be delivered to the interior region through a guide tube that has an inner diameter;
the second elongated member has a second delivery state diameter and is configured to be delivered to the interior region through the guide tube; and
a sum of the first and second delivery state diameters is less than the inner diameter.

46. The bone anchor substrate of claim 43 wherein:
the first elongated member has a first longitudinal axis;
the second elongated member has a second longitudinal axis; and,
when the first and second elongated members are deployed in the interior region, the first and second longitudinal axes are substantially parallel.

47. The bone anchor substrate of claim 43 wherein, when the bone anchor substrate has a central axis:
the first elongated member has a first longitudinal axis;
the second elongated member has a second longitudinal axis; and,
when the first and second elongated members are expandable, in the interior region, the first and second longitudinal axes are substantially conically arranged about the central axis.

48. The bone anchor substrate of claim 43 wherein:
the first web includes a first anchor receiving feature;
the second web includes a second anchor receiving feature; and
the first and second anchor receiving features are sufficiently aligned with each other to engage a bone anchor that penetrates a fragment of the bone.

49. The bone anchor substrate of claim 48 wherein the first and second elongated members are members of a group of elongated members, each member of the group being configured to be deployed alongside another member of the group in the interior region of the bone.

50. The bone anchor substrate of claim 49 wherein a first member of the group is configured to transmit load from a first bone fragment to a second bone fragment via a second member of the group.

51. The bone anchor substrate of claim 50 wherein the first and second members of the group communicate load with each other via a surface contact.

52. The bone anchor substrate of claim 50 wherein the first and second members of the group communicate load with each other via a coupling.

53. The bone anchor substrate of claim 50 wherein the first and second members of the group communicate load with each other via an anchor.

54. The bone anchor substrate of claim 43 further comprising a coupling that is configured to resist distancing of the second elongated member from the first elongated member in response to a force.

55. The bone anchor substrate of claim 43 wherein the coupling is configured to resist the distancing during traversal of the first elongated member and the second elongated member by a bone anchor.

56. The bone anchor substrate of claim 43 wherein the coupling is configured to resist the distancing during loading of the first elongated member and the second elongated member by a bone anchor.

57. The bone anchor substrate of claim 43 wherein a one of the first elongated member and the second elongated member is expandable.

58. The bone anchor substrate of claim 43 wherein a one of the first elongated member and the second elongated member has a radius that varies along the length of the elongated member.

59. The bone anchor substrate of claim 43 wherein the first anchor receiving features include an open cell in a web of open cells.

60. The bone anchor substrate of claim 43 wherein the first anchor receiving features include a closed cell in a web of closed cells.

61. The bone anchor substrate of claim 43 the first anchor receiving features include a tubular portion that defines an anchor receiving slot.

62. The bone anchor substrate of claim 43 wherein the first anchor receiving features include a tubular portion that defines an anchor receiving hole.

63. The bone anchor substrate of claim 43 further comprising a plurality of elongated members wherein the coupling is further configured to resist distancing of each of the plurality of elongated members, the first elongated member and the second elongated member from another of the plurality of elongated members, the first elongated member and the second elongated member.
